SEQUENCE LISTING (41 sequences)

SEQ ID NO: 1 - protein, 41 aa, Hepatitis B virus
MQWNSAAAAAALEHIGPGRAFVVPLEEAWDPSRIGDPALNM

SEQ ID NO: 2 - protein, 9 aa, Homo sapiens
KTWGQYWQV

SEQ ID NO: 3 - protein, 9 aa, Homo sapiens
ITDQVPFSV

SEQ ID NO: 4 - protein, 9 aa, Homo sapiens
YLEPGPVTA

SEQ ID NO: 5 - protein, 10 aa, Homo sapiens
LLDGTATLRL

SEQ ID NO: 6 - protein, 10 aa, Homo sapiens
VLYRYGSFSV

SEQ ID NO: 7 - protein, 9 aa, Homo sapiens
AAGIGILTV

SEQ ID NO: 8 - protein, 9 aa, Homo sapiens
ILTVILGVL

SEQ ID NO: 9 - protein, 9 aa, Homo sapiens
MLLAVLYCL

SEQ ID NO: 10 - protein, 9 aa, Homo sapiens
YMDGTMSQV

SEQ ID NO: 11 - protein, 9 aa, Homo sapiens
YMNGTMSQV

SEQ ID NO: 12 - protein, 9 aa, Homo sapiens
VLPDVFIRC

SEQ ID NO: 13 - protein, 9 aa, Homo sapiens
FLWGPRALV

SEQ ID NO: 14 - protein, 9 aa, Homo sapiens
YLSGANLNL

SEQ ID NO: 15 - protein, 9 aa, Homo sapiens
RMPEAAPPV

SEQ ID NO: 16 - protein, 9 aa, Homo sapiens
STPPPGTRV

SEQ ID NO: 17 - protein, 9 aa, Homo sapiens
LLGRNSFEV

SEQ ID NO: 18 - protein, 9 aa, Homo sapiens
KIFGSLAFL

SEQ ID NO: 19 - protein, 9 aa, Homo sapiens
IISAVVGIL

SEQ ID NO: 20 - protein, 8 aa, Homo sapiens
TLGIVCPI

SEQ ID NO: 21 - protein, 9 aa, Influenza virus
GILGFVFTL

SEQ ID NO: 22 - protein, 13 aa, Hepatitis B virus
TPPAYRPPNAPIL

SEQ ID NO: 23 - protein, 9 aa, Human immunodeficiency virus type 1
ILKEPVHGV

SEQ ID NO: 24 - protein, 92 aa, Homo sapiens
AAGIGILTVFLWGPRALVMELAVLYCLLLDGTATLRLKTWGQYWQVYMDGTMSDVITDQVPFSVYLEFGPVTAILTVILGVLVLPDVFIRCV

SEQ ID NO: 25 - protein, 9 aa, Human immunodeficiency virus type 1
  VARIANT (9): S9L MUTANT EPITOPE
SLYNTVATL

SEQ ID NO: 26 - protein, 9 aa, Human immunodeficiency virus type 1
  VARIANT (9): T9V MUTANT EPITOPE
TLNAWVKVV

SEQ ID NO: 27 - protein, 9 aa, Human immunodeficiency virus type 1
  VARIANT (9): E9V MUTANT EPITOPE
EMMTACQGV

SEQ ID NO: 28 - protein, 10 aa, Human immunodeficiency virus type 1
  VARIANT (10): L10V MUTANT EPITOPE
LLDTGADDTV

SEQ ID NO: 29 - protein, 9 aa, Human immunodeficiency virus type 1
  VARIANT (9): A9M MUTANT EPITOPE
ALVEICTEM

SEQ ID NO: 30 - protein, 11 aa, Human immunodeficiency virus type 1
VLDVGDAYFSV

SEQ ID NO: 31 - protein, 9 aa, Human immunodeficiency virus type 1
  VARIANT (9): V9L MUTANT EPITOPE
VIYQYMDDL

SEQ ID NO: 32 - protein, 9 aa, Human immunodeficiency virus type 1
  VARIANT (9): I9V MUTANT EPITOPE
ILKEPVHGV

SEQ ID NO: 33 - protein, 9 aa, Human immunodeficiency virus type 1
  VARIANT (9): P9L MUTANT EPITOPE
PLVKLWYQL

SEQ ID NO: 34 - protein, 11 aa, Human immunodeficiency virus type 1
  VARIANT (11): E11Q MUTANT EPITOPE
ESELVNQIIEQ

SEQ ID NO: 35 - protein, 10 aa, Human immunodeficiency virus type 1
  VARIANT (10): E10L MUTANT EPITOPE
ELVNQIIEQL

SEQ ID NO: 36 - protein, 9 aa, Human immunodeficiency virus type 1
  VARIANT (9): L9V MUTANT EPITOPE
LLWKGEGAV

SEQ ID NO: 37 - protein, 9 aa, Human immunodeficiency virus type 1
  VARIANT (9): K9L MUTANT EPITOPE
KLTPLCVSL

SEQ ID NO: 38 - protein, 9 aa, Human immunodeficiency virus type 1
  VARIANT (9): K9L MUTANT EPITOPE
KLTPLCVTL

SEQ ID NO: 39 - protein, 9 aa, Human immunodeficiency virus type 1
  VARIANT (9): R9V MUTANT EPITOPE
RLRDLLLIV

SEQ ID NO: 40 - protein, 10 aa, Human immunodeficiency virus type 1
  VARIANT (10): P10L MUTANT EPITOPE
PLTFGWCFKL

SEQ ID NO: 41 - protein, 9 aa, Human immunodeficiency virus type 1
  VARIANT (9): A9L MUTANT EPITOPE
AFHHVAREL

(19) United States
(12) Patent Application Publication
(10) Pub. No.: US 2002/0164721 A1
(43) Pub. Date: Nov. 7, 2002

(54) Design of a polyepitopic construct for the induction of HLA-A2.1 restricted HIV 1 specific CTL responses using HHD mice

(75) Inventors: Huseyin Firat, Paris (FR); Francois Lemonnier, Bourg-la-Reine (FR); Pierre Langlade-Demoyen, Paris (FR); Marie-Louise Michel, Paris (FR)

Correspondence Address: FINNEGAN, HENDERSON, FARABOW, GARRETT &, DUNNER LLP, 1300 I STREET, NW, WASHINGTON, DC, 20005, US

(73) Assignee: Institut Pasteur

(21) Appl. No.: 10/106,487

(22) Filed: Mar. 27, 2002

Related U.S. Application Data
(63) Continuation of application No. 09/675,673, filed on Sep. 29, 2000, now abandoned.
(60) Provisional application No. 60/158,356, filed on Oct. 12, 1999.

(51) Int. Cl. A61K 31/70; A01N 43/04; C12P 21/06; C12P 19/34; A61K 39/29; C12N 15/00; C12N 15/09; C12N 15/63; C12N 15/70; C12N 15/74

(52) U.S. Cl. 435/91.1; 435/91.33; 435/69.1; 424/227.1; 435/320.1; 514/44

(57) ABSTRACT

H-2 class I negative, HLA-A2.1 transgenic HHD mice were used for a comparative evaluation of the immunogenicity of HLA-A2.1 restricted human tumor-associated CTL epitopes and HIV 1-derived epitopes. A hierarchy was established among these epitopic peptides injected into mice in IFA which correlates globally with their capacity to bind and stabilize HLA-A2.1 molecules. A tyrosine substitution in position 1 of the HIV 1-derived epitopic peptides, which increases both their affinity for and their HLA-A2.1 molecule stabilizing capacity, was introduced in a significant proportion of them. DNA immunizations were performed using a construct comprising nucleic acids encoding the epitopes inserted into the pre-S2 segment of the hepatitis B middle glycoprotein. CTL responses against most of the inserted epitopes could be elicited simultaneously in a single animal.

### CROSS-REFERENCE TO RELATED APPLICATION

This application is based on, and claims the benefit of, U.S. Provisional Patent Application Serial No. 60/158,356, filed on Oct. 12, 1999, the entire disclosure of which is hereby incorporated herein by reference.

### BACKGROUND OF THE INVENTION

1. Field of the Invention

This invention relates to polynucleotides and vectors, to compositions containing the polynucleotides and vectors, to polypeptides and polypeptide compositions, and to use of these.

2. Description of the Related Art

CTL-mediated protection against tumors has been documented in mouse experimental models (1). In view of our refined comprehension of the molecular structures recognized by CTL, the search for human tumor-derived CTL epitopes has been undertaken in many laboratories, as best exemplified for melanomas (2). Subsequently, clinical trials using peptide-based immunization protocols provided encouraging results (3). However, the selection of peptide(s) and vaccine strategy remains difficult in view of the number of candidate peptides and variety of immunization strategies. An animal model allowing a controlled evaluation of the immunogenic potential of the epitopic peptides and of the immunization strategies would be of interest before human immunotherapeutic trials.

Classical HLA class I transgenic mice (which still express their own H-2 class I molecules) have been derived to evaluate the immunogenic potential of the identified epitopes and compare the efficiency of different vaccine strategies (4, 7, 44). For example, during the last two decades, many laboratories have devoted themselves to the search for HIV-derived CD8 epitopic peptides, in particular presented by the HLA-A2.1 molecules, which are the most prevalent HLA class I alleles in the Caucasian population. However, unless the third domain of the human molecules was substituted with the corresponding mouse domain, the peripheral CTL repertoire of the transgenic mice was inefficiently mobilized by the transgenic molecules due to poor interaction with mouse CD8 molecules (5). Accordingly, improved usage of HLA class I molecules has been documented in transgenic strains expressing chimeric constructs (a1, a2 human, and a3 mouse) and exploited for the study of CTL responses against certain viral and tumor epitopic peptides (6, 7). Nevertheless, in such mice we observed a profound bias in favor of H-2 restricted CTL responses, and development of CTL responses against a limited number of these epitopes (H. Firat, unpublished observations). To circumvent that bias, we derived a strain of mice in which the H-2 D^{b} and mouse β2-microglobulin (β2m) genes have been disrupted by homologous recombination, and which expresses a chimeric (a1, a2 human, and a3 mouse) HLA-A2.1 heavy chain covalently linked at its N terminus to human β2m light chain. The chimeric protein was designated as the HHD molecule. In HHD transgenic mice, the transgenic molecules are the only class I molecules serologically detectable on cell surfaces and are used more efficiently by CTL in responses against viruses than classical HLA transgenic mice (8).

There is accumulating evidence that cytolytic response is the major effector arm against HIV infection. The viremia of the primo-infection stage starts to decline with the onset of the cytolytic response, weeks before the appearance of neutralizing antibodies (33, 34, 35). The early development in certain individuals of polyepitopic cytolytic responses is associated with a slower disease progression, as well documented in infected newborns (36, 37). In SIV primo-infection of macaques, depletion of CD8³⁰ T cells is immediately followed by an important increase in viremia (38). However, despite the development in most patients of potent CTL responses, in the absence of antiviral drug therapy, the infection usually inexorably progresses towards the AIDS stage, with some noticeable exceptions, in particular patients homozygous for the Δ CCR5 deletion (39).

In the natural course of the HIV infection, the development of cytolytic responses takes, however, a certain number of weeks, with significant differences among individuals depending, in part, on the HLA class I alleles expressed by the patients. Noticeably, HLA-A2.1 responses are significantly delayed, leaving time for the virus to hide as a provirus and escape immune detection (40). Since the initial viral burden has been identified as an important prognosis factor (41, 42), one should expect that a more rapid onset of the appropriate cytolytic responses would significantly influence the HIV 1 infection prognosis. Such an expectation is reinforced by recent reports indicating that cytolytic memory T cell survival is independent of continuous exposure to cognate antigens (43). Altogether, these arguments encourage the continued search for an anti-HIV vaccine.

### SUMMARY OF THE INVENTION

In one aspect, this invention relates to the use of HHQ transgenic mice to compare the immunogenic potential of HLA.-A2.1-restricted human tumor-associated CTL epitopes and different strategies of immunization.

In another aspect, this invention relates to the use of HHQ transgenic mice to evaluate changes in the immunogenic potential of HIV 1-derived CTL epitopes induced by modifying the epitopes. In particular, this aspect of the invention relates to use of HHQ transgenic mice to evaluate modifications of epitopes that improve immunogenicity without altering antigenicity, and to test the capacity of a polyepitopic construct to induce, simultaneously in an individual mouse, cytolytic responses against several HIV 1-derived epitopes. Accordingly, this aspect of the invention relates to polynucleotides and vectors encoding, and polypeptides comprising, at least one HIV 1 epitope that induces a cytolytic response.

Accordingly, this invention provides polynucleotides containing at least part of the coding sequence of the middle glycoprotein of the hepatitis B virus in which is inserted a DNA sequence coding for at least one epitope comprising at least one epitope of a tumor, viral, bacterial, and/or fungal antigen.

This invention also provides a polynucleotide containing at least a part of the preS2 sequence of the genome of HBV, in which is inserted a DNA sequence coding for an epitope comprising at least one epitope of a tumor, viral, bacterial, and/or fungal antigen; and a nucleotide sequence coding for the surface antigen of HBV. The epitope(s) can comprise 1 to 30 epitopes, identical or different, and in a wild type or in a mutated configuration.

This invention also provides a composition containing the polynucleotide sequence of the invention for inducing an in vivo immune response against tumor, viral, bacterial, and/or fungal specific antigens or tissue specific antigens.

This invention also provides a vector for induction of an in vivo cellular or humoral immune response. The vector can comprise the polynucleotide of the invention and an early CMV promoter; preS2 and S nucleotide sequences encoding preS2 and S antigens of HBV; nucleotide sequences derived from the genome of HBV containing postranscriptional regulatory elements (PRE) and allowing nuclear export of RNA corresponding to nucleotide 1,151 to nucleotide 1,684 of the HBV genome, and signal sequences for polyadenylation of messenger RNAs of HBV located at position 1,921 to 1,955 of the HBV genome; and nucleotide sequences of tumor, viral, bacterial, and/or fungal epitopes surrounded up and down by alanine spacers. The vector can also have nucleotide sequences encoding a B cell epitope, which allows the detection of the hybrid proteins, wherein the B cell epitope sequences are fused to the tumor, viral, bacterial, or fungal sequences.

In addition, this invention provides a process of treatment in vivo characterized by construction of a recombinant or synthetic polynucleotide sequence according to the invention; injection of the composition according to the invention into a host; and, optionally, evaluation of cytotoxic response in host lymphocyte population.

Further, this invention provides a composition comprising a hybrid preS2-S protein containing tumor, viral, bacterial, and/or fungal antigens (or epitopes) capable of inducing in vivo a CTL response against several epitopes of one or more antigen. The hybrid proteins in the composition can contain also a tag B cell epitope.

Further, this invention provides a recombinant particle comprising this composition and the small envelope protein of HBV.

Further, this invention provides a process of treating cells of a host characterized by contacting the recombinant particles of the invention with the host's cells, and reinjection of treated cells in the host.

The PRE sequences are published in J. Virology, 1996, pp. 4345-4351 [Donello et al.], the entire disclosure of which is relied upon and incorporated by reference herein. The cell epitope has a minimum of 5 amino acids. The CTL epitope has a minimum of 9 amino acids. The T helper epitope has a minium of 12 amino acids.

### BRIEF DESCRIPTION OF THE DRAWINGS

This invention will be described in detail with reference to the drawings in which:

FIG. 1: Representation of the pCMV-B10 constructs and CTL responses of HHD mice injected with pCMV-B10 recombinant DNA coding a melanoma-based polyepitope.

A. Monoepitopic Constructs. The pCMV-B10 vector is a pcDNA3 derivative (INVITROGEN, Costa Mesa, CA) in which the largely overlapping coding and totally overlapping 3′ untranslated nucleotide sequences for hepatitis B virus middle (and, initiation at ATG 900, termination at TAA 1744) and small (initiation at ATG 1066, termination at TAA 1744) envelope proteins have been inserted downstream of a human CMV immediate early promotor (P_{cmv}). The central part of the coding sequence for the preS2 segment was replaced by a polylinker and the coding sequence for a HIV 1-derived (MN isolate) V3 loop tag. The EcoRI and XhoI restriction sites were used to insert oligonucleotides coding for the selected epitopes.

Amino acid sequence of the modified preS2 segment: MQWNSAAA[epitope]AAA LEHIGPGRAFVVPLEEAWDPSRIGDPALNM (SEQ ID NO: 1). The residual preS2 residues are in bold characters and the V3 loop tag residues in italics. The shaded area corresponds to the introduced epitopic peptide with alanine spacers, other residues originate from the residual polylinker nucleotides except the C terminal methionine, which corresponds to position 1 of the hepatitis B virus small envelope protein.

B. Amino-acid sequence of the melanoma polyepitope.

C. CTL responses against HHD-transfected RMAS cells. Spleen cells of immunized mice were restimulated in vitro as indicated in the legend of Table 4. On day 6, cells were assayed against targets loaded with relevant [(NA17-A.nt38); Melan-A/MART-1.27; gp100.154; gp100.457; Tyrosinase.368-D] and control inf.m.58 peptides. No specific lysis was obtained for MAGE-3.271, Tyrosinase.1, gp100.209, gp100.280, Melan-A/MART-1.32 epitopic peptides.

D. CTL responses against HHD-transfected HeLa target cells producing endogenously the melanoma polyepitope.

FIG. 2 is a graph, in two parts, depicting percent specific lysis vs. E/T ratios.

FIG. 3 is a plasmid map of plasmid pCMV-B10, which is useful in this invention.

FIG. 4 depicts the results of in vitro restimulation and cytolytic activity assays using spleen cells from primed mice that were restimulated with irradiated peptide-loaded, LPS-induced HHD lymphoblasts.

FIG. 5 depicts the CTL response of HHD mice to polyepitopic DNA immunization.

### DETAILED DESCRIPTION OF THE INVENTION

In general, the invention relates to new recombinant HBS particles and nucleotide sequences useful for in vivo anti-tumor therapy, immunization against tumor antigens with a nucleotide sequence or a vector encoding tumor epitopes and preS or preS2-S or S antigen of HBV. The invention also relates to new particles comprising the S antigen of HBV preS2-S and tumor peptides (or antigens). In one embodiment, this invention is related to the construction of a recombinant DNA containing 10 melanoma epitopes inserted in a plasmid (pCMV-B10), which also expresses the pre-S2 and HBS Ag.

In embodiments, the invention provides a polynucleotide containing at least a part of the i coding sequence of the middle glycoprotein of the hepatitis B virus (HBV) in which is inserted a DNA sequence coding for an epitope comprising at least one viral, fungal, bacterial, and/or tumor epitope of a tumor antigen, capable of inducing a cellular response. In embodiments, this aspect of the invention provides: 1) a polynucleotide containing at least a part of the preS2 sequence of the genome of HBV in which is inserted a DNA sequence coding for an epitope comprising at least one tumor, viral, bacterial, or fungal epitope or a tumor, viral, bacterial, or fungal antigen; and 2) a nucleotide sequence coding for the surface antigen of HBV.

In embodiments, the polynucleotide encodes 1 to 30 epitopes, which can be identical or different. The epitopes can be in a wild-type or in a mutated configuration.

The immunization of mice with this naked DNA elicited a very good CTL response against the melanoma polyepitope. The plasmid pCMV-B10 (FIG. 3) is derived from the plasmid pCMVHB-S2-S (CNCM I-1410) described in PCT/FR 9400483 (WO 95-11307) corresponding to U.S. application Ser. No. 633,821, filed on Apr. 27, 1994, the entire disclosures of which are relied upon and incorporated by reference herein.

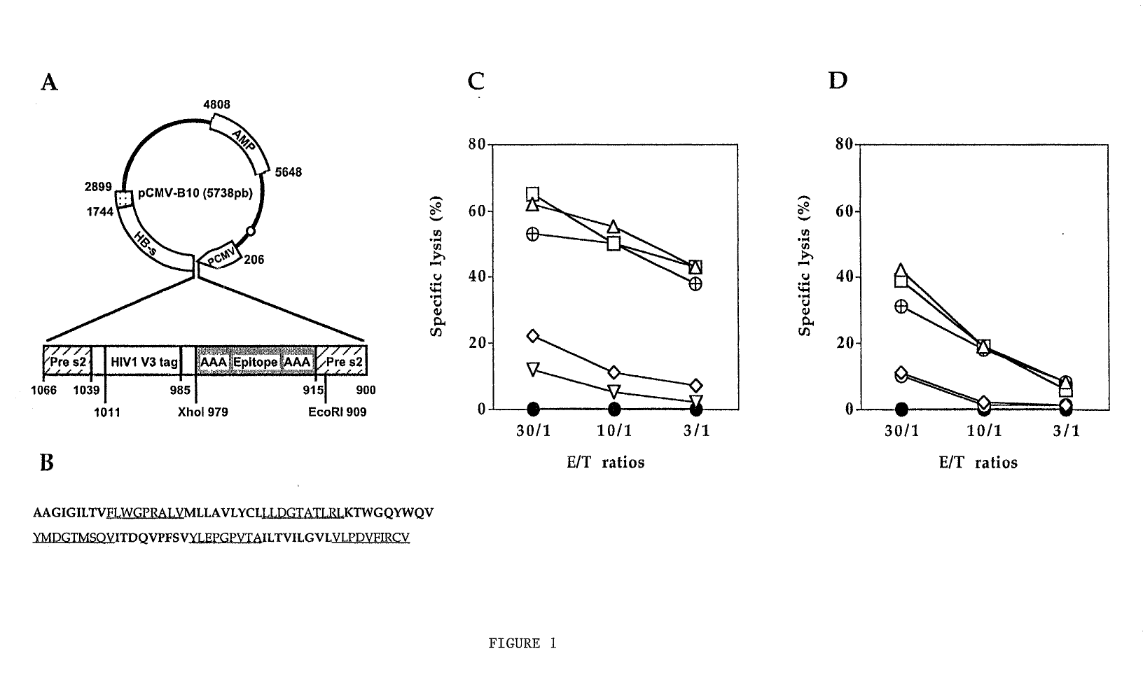

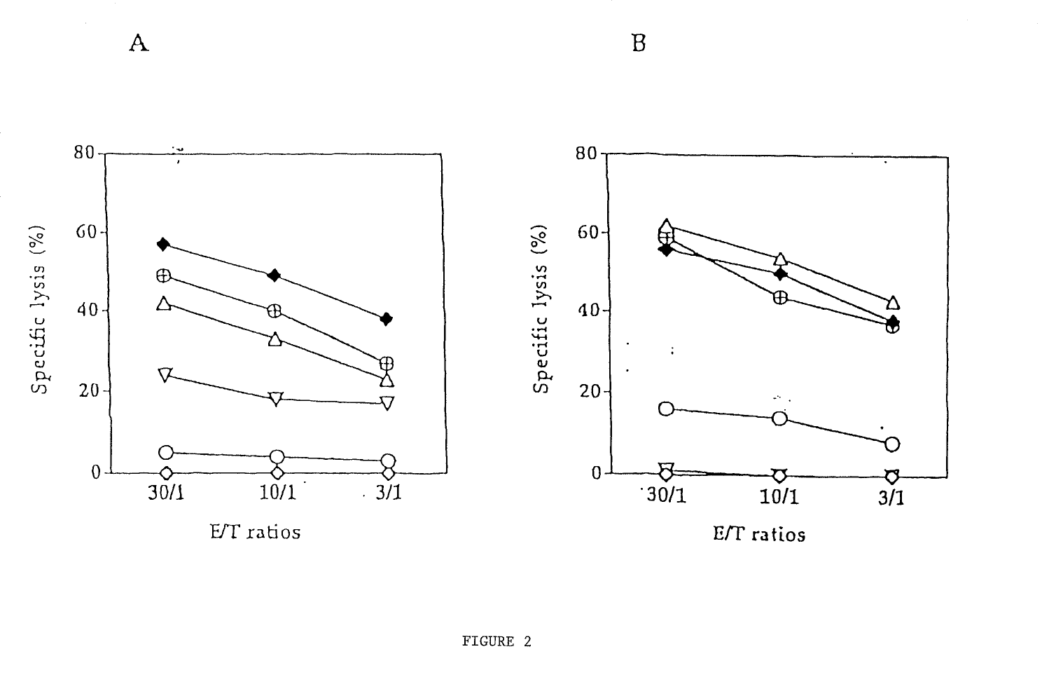

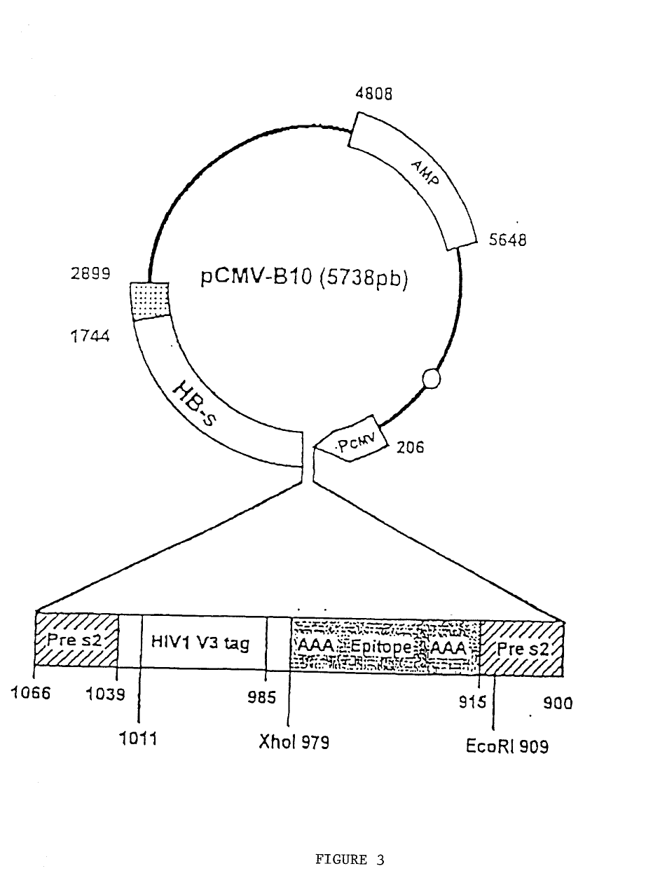

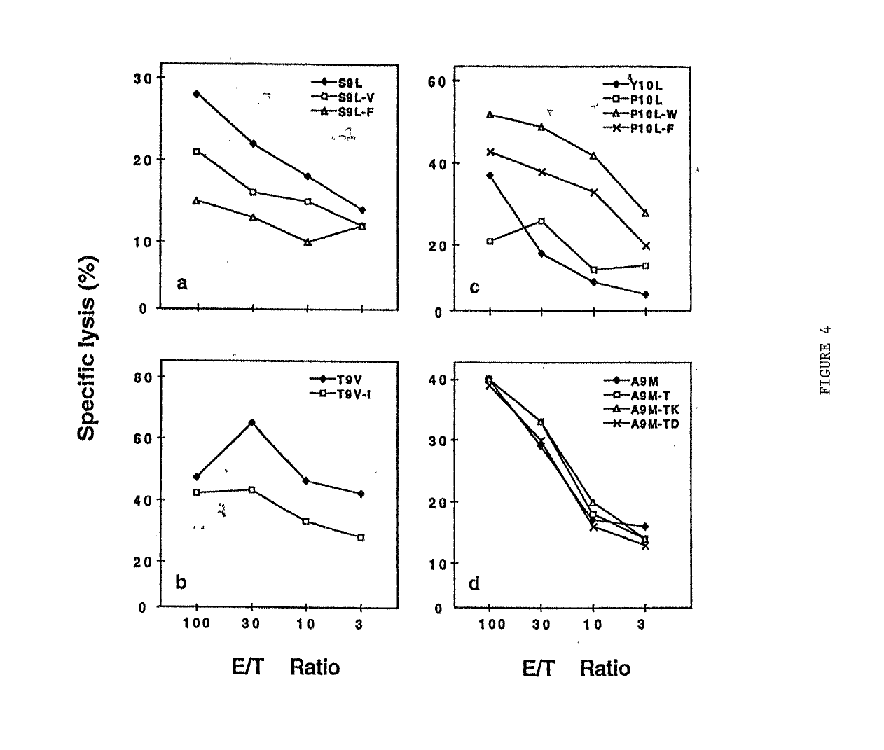

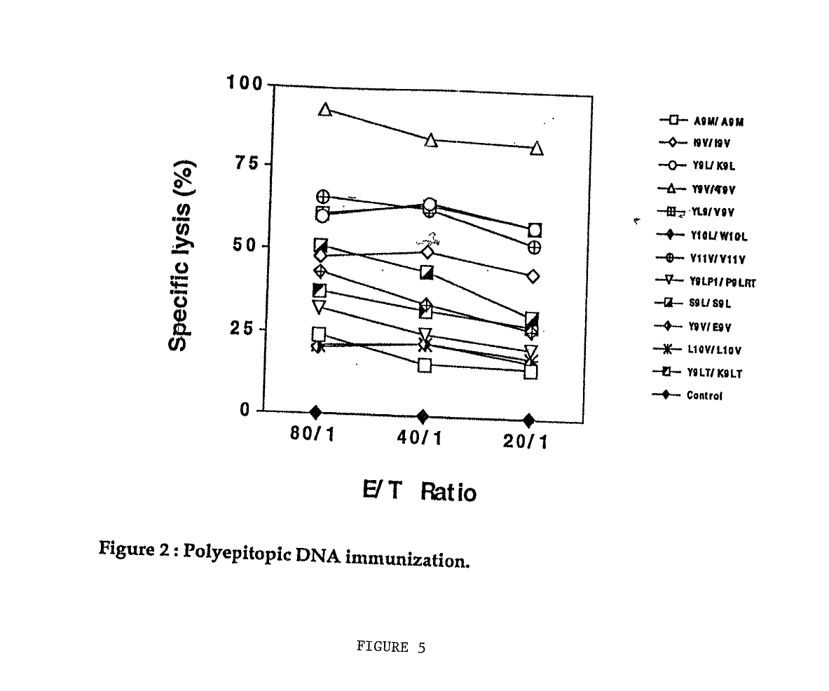

The HIV1-V3 Loop inserted between nucleotides 1,011 and 985 is used as a marker of expression. An insert of particular interest (in case of melanoma) is located at the XhoI site (nucleotide 979) and nucleotide 915 as shown in FIG. 1. The HIV1-V3 loop can be replaced by any B cell epitope, which can be visualized. The size of the insert in this plasmid is very flexible.

One polyepitope has been published by Firat et al. in Eur. Journal of Immunology 29:1-6, 1999, the entire disclosure of which is relied upon and incorporated by reference herein, but the 10 expression is obtained in a recombinant poxvirus vaccine, and the poxvaccine vectors are less safe than pre S2-S HBV particles. (Described in U.S. Pat. Nos. 5,314,808 and 5,591,638). A construction with the vector pCMV-S2-S and V3 loop of gp120 of HIV-1 has been published in 1998 (Virology 240:304-315, 1998), the entire disclosure of which is relied upon and incorporated by reference herein. The details for the construction of pCMV-B10 are disclosed herein. Also relevant to the invention is the hepatitis B virus post transcriptional regulatory element composed of two sub-elements described in J. Virol. 70:4345-4351 (July 1996), the entire disclosure of which is relied upon and incorporated by reference herein.

The invention also provides a composition containing the nucleotide sequence of the invention, which is capable of inducing in vivo an immune cellular response against a viral, a bacterial, a fungal, and/or a tumor specific antigen, tissue specific antigens, and all the self mutated or self-expressed proteins.

Further, a vector for induction of an in vivo cellular or/humoral immune response is provided. The vector comprises: 1) an early CMV promoter, preS2 and S nucleotide sequences encoding preS2 and S antigens of HBV; 2) nucleotide sequences derived from the genome HBV containing postranscriptional regulatory elements (PREs) and allowing nuclear export of RNA corresponding to nucleotide, 1,151 to nucleotide 1,684 of the HBV genome; 3) signal sequences for polyadenylation of messenger RNAs of HBV located at position 1,921 to 1,955 of the HBV genome; and 4) nucleotide sequences of tumor, viral, bacterial, and/or fungal epitopes or antigens surrounded up and down by alanine spacers. In embodiments, the vector also includes nucleotide sequences encoding a B cell epitope, which allows the detection of the hybrid proteins, said B cell epitope sequence being fused to the viral, bacterial, fungal, and/or tumor sequences.

In an aspect of the invention, a process of treatment in vivo is provided. The process comprises: 1) construction of a recombinant or synthetic sequence according to the invention; 2) injection of a composition according to the invention to a host; and, if necessary, 3) a test of evaluation of the cytotoxic responses in hosts lymphocytes population.

In a further aspect of the invention, a composition comprising a hybrid preS2-S protein containing viral, bacterial, fungal, and/or tumor antigens (or epitopes) capable of inducing an in vivo CTL response against several epitopes of one or more bacterial, fungal, viral, and/or tumor antigens is provided. In embodiments, the composition comprises hybrid proteins that also contain a tag B cell epitope. In embodiments, the invention provides recombinant particles comprising the composition according to the invention and the small envelope protein of HBV. In embodiments, the composition of the invention can be characterized as having a viral epitope which is an HIV epitope. The HIV epitope can be chosen from among the envelope protein or the gag, pol, or nef antigens. In embodiments, the composition induces an in vivo cellular immune response against an HIV antigen such as an envelope, gag, pol, or nef protein.

An aspect of the invention provides a process of treatment of cells of a host comprising: 1) contacting the recombinant particles according to the invention with the host's cells; and 2) reinjection of treated cells in the host.

The possibility to introduce, without real size limitations, DNA inserts in the preS2 segment of the middle hepatitis B glycoprotein coding sequence will be exploited to further insert epitopes presented by other HLA class I alleles.

The two first selected (HLA-A3.1, EMBO J. 1984, 3, 887-894 and HLA-B7.2, PNAS, 1990, 87, 2833-2837) should result in polyepitopes of vaccinal interest for more than 80% of the Caucasian human population.

More particularly, in one aspect of the invention, the inventors used H-2 class I negative HLA-A2.1 transgenic HHD mice to evaluate the immunogenic potential of 19 human tumor-associated CTL epitopes and to compare different immunization strategies. A parallel study of the CTL responses of H-2 positive A2A2K^{b} classical transgenic mice illustrated the improved capacity of HHD transgenic mice to develop HLA-A2.1-restricted CTL responses. This advantage was previously documented by analyzing antiviral responses (8) but could have resulted from the preferential development in classical transgenic mice of H-2-restricted CTL responses against other viral epitopes. This explanation cannot apply to the responses induced by HLA-A2.1-restricted synthetic peptides. The present results suggest that the size of the HLA-A2.1-educated CD8⁺ peripheral T cell repertoire is larger in HHD than in A2A2K^{b} transgenic mice, in spite of a 10-fold lower cell surface expression of the transgenic HHD molecules and 5-fold lower CD8⁺ T cell number in the periphery (data not shown). The HHD and A2A2K^{b} molecules being functionally equivalent in terms of antigen presentation (8), we have to postulate that in classical transgenic mice, the co-expression of the A2A2K^{b} and H-2 class I molecules at the thymic level results in preferential H-2-education. This could be due to the fact, documented for HLA-A2.1 molecules, that residues in the second domain of the heavy chain and some of β2m, participate in the CD8 accessory interaction (23). Assuming the affinity of the mouse CD8 molecules to remain higher for H-2 than for chimeric A2A2K^{b} class I molecules, and the thymic education to be a saturable process, one can conceive the quantitative advantage, in terms of HLA education, that would result from the absence of H-2 class I molecules.

Immunizing HHD mice with human tumor-derived CD8 epitopic pepticles alone in IFA, lead us to devise a hierarchy which correlates, although imperfectly, with their binding and stabilizing capacities of HLA-A2.1 molecules. One might wonder whether this hierarchy also applies to humans. It appears unlikely that TcR differences between human and mouse could be of any significant influence considering the huge diversity of the T cell repertoire in both species and the absence of species-specific structural features in the variable TcR segments (24). Self-tolerance and accumulated phylogenic protein differences are more likely to modulate in a species specific manner this epitopic hierarchy. However, the human genetic polymorphism should also, to some extent, have the same consequences at the individual level. Using antigenic formulations which need to be processed, such as Ty-VLP and recombinant HBs particles, one should finally consider the possibility that the cell processing machinery of mouse and man could be functionally different. One such difference concerns the TAP pumps, the human one transporting more efficiently than its mouse counterpart peptides with positively charged C-terminal residues (25). This is of no relevance for HLA-A2.1 transgenic mice, since this molecule binds peptides with leucine or methionine C-termini which are efficiently transported by the mouse TAP pump (26). In fact, so far, all reported observations, except those concerning TAP, suggest a large functional redundancy between the mouse and human processing machineries (6, 27).

The potent CTL responses induced either by Ty-VLP or recombinant pCMV-B10 (HBs) DNA were anticipated. Particulate antigens, which should also be released in the organism after i.m. injection of the recombinant HBs DNA, are good immunogens (41). Endosomal processing of the p1 and HBs proteins included in these particulate antigens might also result in the production of helper pepticles which facilitate the development of cytotoxic responses. Of special interest was the possibility of simultaneously inducing, in a single mouse, CTL responses against 5 different peptides included in the melanoma-based polyepitopic construct. This should compensate for the differences in expression of the molecules of immunological interest among melanomas (9) and reduce the risk of tumor escape. Among the 5 epitopic peptides for which CTL responses were documented, three (gp100.154, NA17-A.nt38, and Melan-A/MART-1.27) are of special interest since they are expressed in 40, 50 and 40%, respectively, of melanomas, with the expression of the NA17-A.nt38 epitope restricted to malignant cells. The 5 epitopes which did not induce CTL responses after polyepitopic immunization are poor binders and poor stabilizers. Modifications are currently tested to enhance their binding and stabilizing capacities and HHD mice are used to verify that the CTL responses they induce cross-recognize the wild-type epitopes.

The weakness of the CTL responses induced by peptide-loaded dendritic cells generated in vitro, was unexpected. This strategy has been documented as very efficient in many situations (28, 29, 30). One explanation could be that HHD mice are not congenic. Minor histocompatibility antigen differences could result in rapid destruction of the injected cells by the recipient mice. Backcrosses are underway to reach a B6 homogeneous genetic background and evaluate such a possibility. Such B6 congenic HHD mice would also provide us with the possibility to evaluate, using EL4 β2 m negative HHD⁺ S3⁻Rob transfectants (8), the protection conferred by the elicited CTL responses.

In a second aspect of the invention, the inventors used H-2 class I negative HLA-A2.1 transgenic HHD mice to evaluate the immunogenic potential of 17 HIV 1 derived, HLA-A2.1 restricted epitopic peptides. The results show that expression of epitopes was achieved and CTL responses against epitopes was elicited. Indeed, CTL responses against multiple epitopes from one construct of the invention could be achieved in a single animal.

Unlike acute viral infections, often characterized by the development of oligoepitopic, immunodominant, CTL response (56), the long-lasting antigenic challenge of an HIV infection is often associated, during the latent chronic phase of the disease, with the development of polyepitopic CTL responses in infected individuals. Whereas such polyepitopic responses during the primo infection stage have been associated with a better prognosis (41, 42), their emergence is usually delayed, reflecting asynchronicity in the development of different responses and variations in their capacity to persist over time. It is likely that such variability reflects, in part, intrinsic differences in the immunogenic potential of the different epitopes. In fact, a significant number of the HIV 1-derived epitopes tested and described herein failed to induce significant CTL responses when injected in IFA to mice. These failures correlate quite satisfactorily with a low affinity for HLA-A2.1 molecules and a low stabilizing capacity of these epitopic peptides.

To be of vaccinogenic interest, the immunogenicity of these epitopes has to be implemented in the respect to their antigenicity. The replacement in P1 of the wild-type residue by a tyrosine has, in most cases, fulfilled these goals. Such an enhancing effect was first documented for the I9V epitopic peptide with human CTL (54). A similar observation was made with a melanoma epitopic peptide (Melan A/MART1.27), in which case the tyrosine residue was added at its N terminus (57). We have generalized these observations to a series of 30 human tumor-derived epitopic peptides (55). The tyrosine enhancement of affinity and stabilizing capacity could be due to the development of stacking interaction with the aromatic rings of tryptophan 17, a type of interaction which has been documented crystallographically for the hepatitis B nucleocapsid 18-27 epitopic peptide with a natural P1 phenylalanine residue (58). With few exceptions (e.g., A9M, V11V), tyrosine substitution did not alter the antigenicity for CTL of the HIV 1 peptides. The lateral position of the P1 residue side-chain in the antigenic surface presented to the TcR (58), and the immediate vicinity of the main anchor residue (Leu in P2 for HLA-A2.1 bound peptides), probably conceal the structural modifications to a limited area only scanned by few CTLs. The tryptophan 167 being conserved among most HLSA class I alleles, one might expect the P1 tyrosine effect to be generalized to other HLA class I molecules. However, the natural structural polymorphism of HLA class I molecules, including that of their major anchor positions, makes it likely that antigenicity of tyrosine-substituted peptides will not be always as well preserved as in the case of HLA-A2.1.

To be of a real value, a vaccine formulation against HIV should not only induce simultaneously CTL responses against several viral epitopic peptides, but also target structurally stable peptide motifs. Alternatively, it should induce responses susceptible to recognize most of their natural variants. These two goals are satisfactorily reached with the polyepitopic construct of the invention. With the exception of the K9L(T) and K9L (79.4% and 8.9%, respectively, of the HIV isolates), in which case the lack of cross-recognition led us to introduce both motifs in the polyepitope, all other cases showed that either the epitopic peptides were highly conserved among isolates or the induced responses cross-recognized the main variants. This implies that, despite the extensive structural variability of the HIV, a polyepitope construct of potential vaccine interest can be designed for HLA-A2.1 individuals. In fact, such a construct has been designed, and designed in such a way that it an be easily implemented, either by the insertion of new HLA-A2.1 restricted epitopic peptides or by the insertion of epitopic peptides corresponding to other HLA class I alleles to cover a larger fraction of the population and make HLA class I typing of the vaccines unnecessary.

DNA immunizations according to this invention are well adapted for experimental and clinical purposes. Their efficiency in primates and feasability on a mass scale in humans are feasible. The HHD mice are certainly well adapted to evaluate other vaccine strategies, as exemplified by another study with recombinant vaccines (59), but the natural resistance of mice to HIV precludes their use for protection experiments, which could only be approached in primate models.

The results obtained with the 19 human tumor-associated CTL epitopes and the 17 HIV 1-derived epitopes shows that the transgenic mice, polynucleotides, and vectors of the invention can be used with a variety of epitopes. Thus, it is apparent that epitopes from other organisms, such as mammals other than humans, viruses other than HIV 1, as well as bacteria and fungi, would work according to the present invention.

### EXAMPLES

This invention will be described in greater detail in the following examples, which are exemplary only, and do not in any way limit the scope of the invention.

Materials and Methods

Mice

HHD mice express a transgenic monochain histocompatibility class I molecule in which the C terminus of the human β2m is covalently linked to the N terminus of a chimeric heavy chain (HLA-A2.1 α1-α2, H-2D^{b} α3-transmembrane, and intracytoplasmic domains). The H-2D^{b} and mouse β2m genes of these mice have been disrupted by homologous recombination resulting in complete lack of serologically detectable cell surface expression of mouse histocompatibility class I molecules (8).

A2A2K^{b} mice were obtained from HARLAN SPRAGUE DAWLEY (Indianapolis, Ind.). These mice express chimeric heavy chain (HLA-A2.1 α1α2, H-2K^{b} α3 transmembrane, and cytoplasmic domains) in non-covalent association with mouse β2m. They additionally express a full set of C57B1/6 derived (H-2^{b}) class 1a and 1b mouse histocompatibility molecules.

All mice used were bred in our animal facility.

Peptides, Lipopeptides, and Immunization Procedures

Peptides, purchased from either NEOSYSTEM (Strasbourg, France) or SYNT:EM (Nimes, France), were dissolved in dimethylsulfoxide (DMSO, 20 μl/mg of peptide) and subsequently diluted in PBS (2 mg/ml). Mice were injected s.c. at the base of the tail with 100 μg of a HLA-A2.1-restricted peptide, with or without 140 μg of a helper peptide, emulsified in IFA (DIFCO, Detroit, Mich.) 7 days before in vitro restimulation.

Lipopeptides were synthesized as already described (21), resulting in covalent linkage of the peptide N terminus to a S-[2.3 palmitoyloxy-(2-R)-propyI]-N-palmitoyl-(R)-cysteine moiety (P3C) via a two serine spacer. Lipopeptides were dissolved in DMSO (20 μl/mg), then diluted in PBS 1×(2 mg/ml). One hundred μg were injected i.p. 2 weeks before in vitro restimulation.

Recombinant HBs DNA Constructs and Immunization Procedure

Synthetic complementary oligonucleotides corresponding to the selected T cell epitopes l were individually inserted into the pre-S2 segment of the hepatitis B surface (HBs) middle protein using a pCMV-B10 mammalian cell-expression vector (FIG. 1A) (20). Each epitope was flanked on both sides by a 3 alanine spacer. An HIV-1 derived V3 loop tag was inserted in the pre-S2 sequence just after the C-terminal alanine spacer. Recombinant plasmids were purified on LPS-free QIAGEN columns (QIAGEN, Hilden, Germany). Mice were injected i.m. with 10 μM cardiotoxine (LATOXAN, Rosans, France) in 50 μl PBS 1× and, 5 days later, with 50 μg of pCMV-B10 DNA for a 21 day priming.

Recombinant Ty-VLP and Immunization Procedure

Construction and purification of recombinant Ty-VLP were performed as previously described using a pOGS40 yeast-expression vector (31). PCR-amplified oligonucleotides corresponding to the selected epitopes were produced from the recombinant pCMV-B10 constructs, in order to include the two 3 alanine spacers and the HIV 1-derived V3 loop tag. They were introduced in frame in a BamHI site at the 3′ end of the coding sequence of the Ty p1 protein. Ty-VLP and purification were monitored by western blotting using mAb F5.5 against the V3 loop tag (HYBRIDOLAB, Institut Pasteur, Paris, France). The hybrid Ty-VLPs were injected (100 μg/mouse) s.c. into mice for a 14 day in vivo immunization.

Polyepitopic Recombinant HBs DNA Construct

In general, polyepitope DNA was constructed in three steps, each step using synthetic partially complementary oligonucleotides (Genset, Paris, France) and PCR amplification. Introducing appropriate restriction sites for ligations, a complete polyepitopic construct was finally inserted between the EcoRI and XhoI sites of the pCMV expression vector. Recombinant plasmids were purified on LPS-free QIAGEN columns (QIAGEN, Hilden, Germany). Mice were injected i.m. with 10 μM cardiotoxin (LATOXAN, Rosans, France) in 50 μl PBS and, 5 days later, with 100 μg of pCMV-B10DNA for a 21 day priming.

For expression of tumor epitopes, a DNA sequence encoding 10 melanoma-derived HLA-A2.1-restricted CTL epitopes (FIG. 1B) was amplified by PCR (Mateo L, in preparation). The polyepitope sequence was inserted in frame between the EcoRI-XhoI sites of the pCMV-B10 expression vector. Immunizations with naked DNA and in vitro restimulations were performed as described above, except that 10% TCGF was added to the culture medium for the last two days of culture.

For expression of HIV 1-derived epitopes, a DNA sequence encoding HIV epitopes was amplified by PCR and inserted between the EcoRI-XhoI sites of the pCMV-B10 vector. In some cases, an HIV 1-derived (MN isolate) V3 loop tag was also inserted, downstream of the polyepitope.

Generation of Dendritic Cells and Immunization Procedure

Bone marrow-derived dendritic cells were obtained as previously described (32) with some modifications. Bone marrow mononuclear cells were cultured in RPMI supplemented with 10% FCS, 2 mM L glutamine, 50 U/ml penicillin, 50 μg/ml streptomycin, 5×10⁵ M 2-mercaptoethanol (complete RPMI medium), further supplemented with 20 ng/ml of recombinant mouse GM-CSF and 100 ng/ml recombinant mouse IL4 (both from GENZYME, Cambridge, Mass.). On days 2 and 6, non-adherent cells were removed, and fresh complete RPMI medium, supplemented with 10 ng/ml mouse GM-CSF and 50 ng Mouse IL4, was added. On day 7, the culture medium was replaced by complete RPMI medium supplemented with 100 U/ml of mouse TNFα. Dendritic cells, collected on day 9, were more than 95% pure (1A^{b+}, HHD⁺, CD3⁻, 33D1⁺, NDL145⁺, and CD 11c⁺) as assessed with appropriate mAb. These dendritic cells were loaded with peptides (2×10⁶ cells/ml, 10 μg/ml of peptides, 2 h at RT in FCS-free RPMI medium), then washed (×3) and injected (1×10⁶ cells/mouse) i.v. into recipient mice for in vivo priming 14 days before in vitro restimulation.

In Vitro Restimulation and Cytolytic Assays

Spleen cells from primed mice were restimulated using irradiated (5000 rads) peptide-loaded (5×10⁶ cells/ml, 10 μg/ml peptide, 2 h at RT in FCS-free RPMI medium), LPS-induced (25 μg/ml LPS, 7 μg/ml dextran sulfate, in complete RPMI medium, 48 h of culture) HHD lymphoblasts. On day 6, cultured cells were tested in a 4 h ⁵¹Cr-release assay, using as targets HHD-transfected TAP⁻RMA-S cells loaded with relevant or negative control influenza matrix 58-66 (Inf.m.58) peptides (10 μg/ml, 5×10⁶ cells/ml, in FCS-free RPMI medium, 2 h at RT). Specific lysis was calculated as follows: (experimental release−spontaneous release)/total release−spontaneous release)×100.

Peptide Binding and Stabilization of HLA-A2.1 Molecules

T2 (TAP³¹ , HLA-A2.1⁺) cells were incubated overnight at 37° C. (1×10⁶ cells/ml) in FCS-free RPMI medium supplemented with 100 ng/mI of human α2m (SIGMA, St Louis, Mo.) in the absence (negative control) or presence of either reference HIV 1 reverse transcriptase 476-484 (HIV 1 rt.476) or tested peptides at various final concentrations (100, 10, 1, and 0.1 μM). Following a 1 h incubation with brefeldine A (0.5 μg/ml, SIGMA), T2 Cells were labeled (30 min, 4° C.) with a saturating concentration of anti-HLA-A2.1 (BB7.2) mAb, then washed twice. The cells were then incubated (30 min, 4° C.) with saturating concentration of FITC-conjugated goat IgG F(ab′)2 anti-mouse Ig (CALTAG, South San Francisco, Calif.), washed (×2), fixed in PBS 1×, 1% paraformaldehyde, and analyzed using a FACs Calibur cytofluorometer (BECTON DICKINSON, IMMUNOCYTOMETRY SYSTEMS, San Jose, Calif.). The mean intensity of fluorescence (MIF), observed for each peptide concentration (after subtraction of the MIF observed without peptide), was used as an estimate of peptide binding. For each peptide, the concentration needed to reach 20% of the maximal binding (as defined with HIV 1 rt.476 peptide) was calculated. Relative affinity (RA) is the ratio of the concentrations of tested and HIV 1 rt.476 reference peptides needed to reach this value. The lower the RA, the stronger the binding. Stabilization assays were performed similarly. Following initial evaluation of peptide binding (t 0), cells were washed in RPMI complete medium to remove free peptides and incubated, in the continuous presence of brefeldine A (0.5 μg/ml) for 2, 4, 6, and 8 h. The amount of stable peptide-HLA-A2.1 complexes was estimated, as described above, by indirect immunofluorescence analysis. The half-life of complexes (DC50) is the time required for a 50% reduction of the t0 MIF value.

Abbreviations Used

β2 m, β2-microglobulin; DC50, Decay complexes 50 (half-life of peptide-HLA-A2.1 complexes); HBs middle protein, Hepatitis B surface middle protein; RA, relative affinity; TAP, Transporter associated with Antigen Presentation; VLP, virus-like particles.

### EXAMPLE 1

Comparative Evaluation of the Immunogenic Potential of Human Tumor-Derived CD8 Epitopic Peptides with HHD and A2A2 K^{b} Mice

Nineteen HLA-A2.1 restricted synthetic epitopic peptides (9, 10, 11, 12) listed in Table 1 were injected s.c. in IFA in at least six H-2 negative HHD mice and six H-2 positive, A2A2K^{b} transgenic mice. Seven days later, spleen cells from each animal were separately restimulated in vitro and then tested against Transporter associated with Antigen Presentation (TAP)-deficient HHD-transfected RMA-S peptide-loaded target cells.

TABLE 1
List of the epitopic peptides tested^{a}

| | | |
|---|---|---|
| Protein | Epitopic peptide | Sequence |

Human melanoma

| | | |
|---|---|---|
| gp100 | 154-162 | KTWGQYWQV (SEQ ID NO:2) |
| | 209-217 | ITDQVPFSV (SEQ ID NO:3) |
| | 280-288 | YLEPGPVTA (SEQ ID NO:4) |
| | 457-466 | LLDGTATLRL (SEQ ID NO:5) |
| | 476-485 | VLYRYGSFSV (SEQ ID NO:6) |
| Melan-A/ | 27-35 | AAGIGILTV (SEQ ID NO:7) |
| MART-1 | | |
| | 32-40 | ILTVILGVL (SEQ ID NO:8) |
| Tyrosinase | 1-9 | MLLAVLYCL (SEQ ID NO:9) |
| | 368-376-D^{b} | YMDGTMSQV (SEQ ID NO:10) |
| | 368-376-N^{b} | YMNGTMSOV (SEQ ID NO:11) |
| NA17-A | nt38-64^{c} | VLPDVFIRC (SEQ ID NO:12) |
| MAGE-3 | 271-279 | FLWGPRALV (SEQ ID NO:13) |

other human tumors

| | | |
|---|---|---|
| CEA | 571-579 | YLSGANLNL (SEQ ID NO:14) |
| p53 | 65-73 | RMPEAAPPV (SEQ ID NO:15) |
| | 149-157 | STPPPGTRV (SEQ ID NO:16) |
| | 264-272 | LLGRNSFEV (SEQ ID NO:17) |
| Her2/neu | 369-377 | XIFGSLAFL (SEQ ID NO:18) |
| | 654-662 | IISAVVGIL (SEQ ID NO:19) |
| HPV16 E7 | 86-93 | TLGIVCPI (SEQ ID NO:20) |
| Viruses | | |
| Inf.m | 58-66 | GILGFVFTL (SEQ ID NO:21) |
| HBVc | 128-140 | TPPAYRPPNAPIL (SEQ ID NO:22) |
| HIV 1 rt | 476-484 | ILKEPVHGV (SEQ ID NO:23) |

^{a}Human melanoma and other tumor epitopic peptides have been reviewed recently (9). Influenza matrix (Inf.m.58), hepatitis B virus core (HBVc) and HIV 1 reverse transcriptase (rt) epitopic peptides are from references 10-12 respectively.
^{b}Asparagine 370 being a glycosylation site, cytosolic deglycosylation results in presentation to CTL of the 368-376 epitopic pepticle with a Aspartate 370 residue.
^{c}Epitopic peptide corresponding to a tumor-specific transcript initiated by a cryptic promotor and resulting in the translation of intronic nucleotides (38 to 64) of the N-Acetyl glucosaminyl-Transferase-V gene.

TABLE 2
CTL responses against tumor epitopic peptides in IFA and
H.A.-A2.1 peptide binding and stabilizing capacities.

| | HHD mice^{a} | | A2A2Kb mice^{a} | | | |
|---|---|---|---|---|---|---|
| Peptide | R/T (lysis in %)^{b} | | R/T (lysis in %)^{b} | | RA^{c} | D50(h)^{d} |
| gp100.154 | 4/6 | (39, 51, 57, 60) | 5/6 | (22, 34, 39, 44, 50) | 2.28 | 6-8 |
| gp100.209 | 1/6 | (23) | 0/6 | | 1.32 | 4 |
| gp100.280 | 3/16 | (13, 23, 47) | 0/6 | | 1.35 | 4 |
| gp100.457 | 0/6 | | 0/6 | | 1.65 | 2-4 |
| gp100.476 | 2/6 | (54, 70) | 1/6 | (29) | 10 | 4-6 |
| Melan-A/MART-1.27 | 2/8 | (15, 19) | 0/6 | | 2.16 | 4 |
| Melan-A/MART-1.32 | 0/6 | | 0/6 | | 21.1 | 2-4 |
| Tyrosinase.1 | 0/10 | | 0/6 | | >60 | 2-4 |
| Tyrosinase.368-D | 1/6 | (10) | 0/6 | | 2.27 | >6 |
| Tyrosinase.368-N | 4/15 | (12, 12, 20, 29) | 0/6 | | 2.2 | >8 |
| NA17-A.nt38 | 4/7 | (15, 23, 25, 30) | 0/6 | | 1.52 | >8 |
| MAGE-3.271 | 1/6 | (33) | 0/6 | | 0.91 | 6 |
| CEA.57-1 | 6/6 | (64, 67, 70, 71, 73, 75) | 0/6 | | 2.8 | >8 |
| p.53.65 | 4/6 | (10, 12, 26, 60) | 0/6 | | 0.91 | 6-8 |
| p53.149 | 0/6 | | 0/6 | | 36.6 | <2 |
| p53.264 | 0/7 | | 0/6 | | 2.09 | 6-8 |
| Her2/neu.369 | 5/6 | (12, 18, 22, 33, 39) | 0/6 | | 2.24 | 6-8 |
| Her2/neu.654 | 1/6 | (42) | 0/6 | | 11 | 4 |
| HPV E7.88 | 0/6 | | 2/6 | (10, 13) | 0.9 | >8 |

^{a}Spleen cells from mice injected s.c. with peptide In IFA 7 days before were in vitro restimulated and assayed 6 days later against HHD-transfected RMA-S cells loaded with relevant or control (inf.m.58) peptides.
^{b}R/T: responder versus tested mice. Mice were considered as responders when at least 10% specific lysis was observed. The values in parenthesis correspond to the maximal lysis observed for each responder mouse, usually at a 60:1 E/T ratio.
^{c}Relative affinity (RA) is the ratio of the concentrations of tested versus reference peptides needed to reach 20% of the maximal amount of stabilized molecules as defined with high concentrations of reference peptide.
^{d}Half-life of stabilized peptide-HLA-A2.1 complexes (DC 50) was evaluated following T2 cells and pepticle overnight incubation by measuring the amount of residual cell surface peptide-HLA-A2.1 complexes at time intervals (0, 2, 4, 6, 8 h) using indirect immunofluorescence and FACS analysis.

Only 3 peptides elicited HLA-A2.1-restricted CTL responses in A2A2 K^{b} mice whereas 12 did so in HHD mice (Table 2). Considering the number of responding mice and the level of specific lysis, a hierarchy could be devised with strong (gp100.154 and CEA.571), intermediate (Tyrosinase.368-N, NA17-A.nt38, p53.65, and Her2/neu.369), weak (gp100.209, gp100.280, gp100.476, Melan-A/MART-1.27, Tyrosinase.368-D, MAGE-3.271, and Her2/neu.654), and inefficient (gp100.457, Melan-A/MART-1.32, Tyrosinase.1, p53.149, p53.264, and HPV E7.86) CTL inducers.

### EXAMPLE 2

HLA-A2.1 Binding and Stabilizing Capacities of the Epitopic Peptides

The immunogenicity of CD8 epitopic peptides largely reflects their binding and stabilizing capacities, with most of the strong CTL-inducers being both good binders and stabilizers (13,14). Using TAP-deficient, HLA-A2.1⁺ T2 cells, we evaluated these parameters in an immunofluorescence assay as indicated in Materials and Methods section.

The results shown in Table 2 demonstrate that as a rule, strong and intermediate CTL-inducers fell into the strong binder, strong stabilizer group (RA<3, DC50>4 h). There were, however, exceptions, most of which concern peptides such as MAGE-3.271, and p53.264 with high binding and stabilizing capacities, but poor CTL-induction capacity. Thus, HLA-A2.1 binding and stabilizing capacities of epitopic peptides correlate well, but not completely, with peptide immunogenicity.

### EXAMPLE 3

Co-immunization with Helper Hepatitis B Virus Core (HBVc 128-140) Peptide

Whereas the sole immunization with class I-restricted synthetic peptides of optimal size is sufficient for the induction of CTL responses in some cases (15), the need for help has been 5 documented in other circumstances (16). Therefore, the CTL responses of HHD mice which express H-2^{b} class II molecules were tested by co-injecting tumor-associated peptides and the IA^{b-} restricted HBVc.128 peptide (11).

Globally, all cytolytic responses were either induced or improved, except in the case of the Her2/neu.654 peptide (Table 3). Peptides of the weak and inefficient CTL-inducer groups (gp100.457, Tyrosinase.1, and MAGE-3.271) elicited good CTL responses in a large proportion of mice when co-injected with the helper peptide and peptides of the strong or intermediate CTL-inducer group (gp100.154, NA17-A.nt38, p53.65 and Her2/neu.368) elicited stronger responses in a larger proportion of mice. However, one noticeable exception, the CEA.571 peptide, turned out to be less immunogenic when co-injected with the helper peptide.

TABLE 3
CTL responses of HHD mice co-immunized in IFA with
HBVC.128 helper peptide^{a}

| Peptide | R/T^{b} | (lysis in %) |
|---|---|---|
| gp100.154 | 9/11 | (43, 59, 60, 64, 77.77, 80, 82, 85) |
| gp100.209 | 3/6 | (31, 36, 56) |
| gp100.280 | 2/8 | (12, 16) |
| gp100.457 | 5/6 | (14, 35, 43, 77, 79) |
| gp100.476 | 6/7 | (20, 22, 59, 63, 75, 79) |
| Melan-A/MART-1.27 | 4/5 | (10, 19, 20, 30) |
| Melan-A/MART-1.32 | 1/6 | (10) |
| Tyrosinase.1 | 5/6 | (27, 33, 40, 42, 51) |
| Tyrosinase.368-N | 5/12 | (21, 36, 70, 72, 78) |
| Tyrosinase.368-D | 2/6 | (13, 15) |
| NA7-A.nt38 | 5/6 | (36, 39, 61, 64, 71) |
| MAGE-3.271 | 6/6 | (34, 38, 59, 63, 64, 79) |
| CEA.571 | 3/6 | (20, 32, 46) |
| p53.65 | 5/6 | (29, 31, 40, 41, 55) |
| p53.149 | 3/6 | (20, 51, 71) |
| p53.264 | 2/3 | (37, 64) |
| Her2/neu.369 | 7/8 | (21, 23, 25, 39, 40, 72, 75) |
| Her2/neu.654 | 0/6 | |
| HPV 16 E7.86 | 2/6 | (34, 48) |

^{a}Spleen cells from mice, co-injected s.c. with CD8 epitopic (50 μg) and helper (140 μg) peptides in IFA 7 days before, were in vitro restimulated with peptide-loaded LPS-lymphoblasts and assayed 6 days later at different E/T ratios against HHD-transfected RMA-S target cells loaded with relevant or control (Inf.m.58) peptides.
^{b}R/T, responder versus tested mice (see table 2^{b})

### EXAMPLE 4

Comparison of Monoepitopic Immunization Strategies

Five peptides, one strong (CEA.571), two intermediate (NA17-A.nt38, Tyrosinase.368-N), and two weak (gp100.280, Tyrosinase.368-D) CTL-inducers were selected for this study. Four immunization strategies were compared: peptide-loaded, in vitro generated dendritic cells (17, 18), yeast-derived hybrid Ty-virus like particles (VLP) (19), recombinant Hepatitis B surface (HBs) middle protein encoding DNA (pCMV-B10-DNA) (20), and lipopeptides (21). Following in vitro restimulation, spleen cells of HHD mice were individually tested in a CTL assay.

Immunization with recombinant Ty-VLP and recombinant pCMV-B10-DNA gave the best results with strong CTL responses against CEA.571, and NA17-A.nt38 T cell epitopes in all animals tested (Table 4). Some responses of weak to strong magnitude could even be elicited against the weak CTL-inducers gp100.280 and Tyrosinase.368-D. Immunization with lipopeptides resulted in CTL responses but with large interindividual differences. Surprisingly, peptide-loaded dendritic cells (95% pure), gave poor results. It is noteworthy that the weak CTL responses observed in this latter case were clearly evidenced following a second in vitro restimulation in the presence of IL2.

TABLE 4
CTL responses against tumor epitopic peptides in IFA and
HLA.-A2.1 peptide binding and stabilizing capacities.

| | | | | Tyrosinase | | Tyrosinase | | NA17- | | | |
|---|---|---|---|---|---|---|---|---|---|---|---|
| | | gp100.280 | | 368-D | | 368-N | | A.nt38 | | CEA.571 | |
| | Mice | lysis in % | | lysis in % | | lysis in % | | lysis in % | | lysis in % | |
| Dendritic cells | 1 | 0 | (0) | 7 | (68) | 15 | (61) | 6 | (7) | 2 | (0) |
| | 2 | 0 | (0) | 23 | (56) | 0 | (1) | 0 | (2) | 4 | (22) |
| | 3 | 0 | (1) | 12 | (64) | 0 | (3) | 0 | (6) | 4 | (1) |
| | 4 | 0 | (0) | 20 | (64) | 0 | (17) | 3 | (5) | 1 | (5) |
| | 5 | 0 | (1) | 8 | (92) | 0 | (30) | 5 | (24) | 1 | (2) |
| | 6 | 0 | (1) | 3 | (71) | 8 | (80) | 3 | (9) | 0 | (0) |
| Ty-VLP | 1 | 8 | | nd | | 0 | | 55 | | 56 | |
| | 2 | 3 | | nd | | 61 | | 39 | | 59 | |
| | 3 | 1 | | nd | | 58 | | 50 | | 51 | |
| | 4 | 8 | | nd | | 39 | | 60 | | 58 | |
| | 5 | 4 | | nd | | 29 | | 59 | | 46 | |
| | 6 | 18 | | nd | | 17 | | 61 | | 62 | |
| pCMV-B10 DNA | 1 | 0 | | 57 | | 6 | | 35 | | 19 | |
| | 2 | 0 | | 55 | | 12 | | 43 | | 66 | |
| | 3 | 8 | | 28 | | 16 | | 47 | | 40 | |
| | 4 | 36 | | 58 | | 13 | | 44 | | 65 | |
| | 5 | 21 | | 56 | | 11 | | 40 | | 62 | |
| | 6 | 9 | | 28 | | 38 | | 54 | | 55 | |
| Lipopeptides | 1 | 0 | | 0 | | 51 | | 0 | | 0 | |
| | 2 | 8 | | 10 | | 20 | | 12 | | 8 | |
| | 3 | 40 | | 1 | | 0 | | 0 | | 8 | |
| | 4 | 8 | | 4 | | 5 | | 68 | | 34 | |
| | 5 | 0 | | 0 | | 11 | | 65 | | 3 | |
| | 6 | 21 | | 0 | | 12 | | 47 | | 0 | |
| Spleen cells from mice previously injected with either peptide-loaded dendritic cells differentiated in vitro, on purified TY-VLP, on naked pCMV-B10 DNA encoding recombinant HBs particles, or lipopeptides were restimulated in vitro using peptide-loaded, irradiated LPS-lymphoblasts. Six days later, they were assayed at | | | | | | | | | | | |
| # different E/T ratios against HHD-transfected RMAS cells loaded with relevant or control (Inf.m.58) peptides. The values correspond to the highest specific lysis observed, usually at a 60/1 E/T ratio. | | | | | | | | | | | |

ND: not done.
Numbers in parentheses: specific lysis following a second in vitro restimulation of effector cells in the presence of 10% TCGF.

### EXAMPLE 5

Melanoma Polyepitopic Immunization

Using a polyepitope construct (22), we evaluated the possibility of simultaneously inducing in a single mouse CTL responses against several melanoma epitopes. Six HHD mice were injected with pCMV-B10 DNA encoding recombinant preS2/S glycoproteins containing a polyepitopic melanoma-derived motif (FIG. 1A and B). Following separate in vitro splenocyte restimulation by each epitopic peptide, they were individually assayed against peptide-loaded HHD-transfected RMA-S cells and HHD-transfected human HeLa cells further transfected with a HIV-1 derived polyepitope expression vector (H. Firat, in preparation).

Whether peptide-loaded or cells expressing endogenously the polyepitopic construct were used as target, specific CTL responses were regularly induced against 4 to 5 out of the 10 melanoma epitopes included in the polyepitopic motif (FIG. 1C and D). Strong responses were elicited against gp100.154 and NA17-A.nt38 epitopic peptides classified as strong and intermediate CTL-inducers, respectively. Significant responses were observed against gp100.457 and Melan-A/MART.1.27 (strong CTL-inducers when associated with a helper peptide). Tyrosinase.368-D or gp100.209 also elicited CTL responses depending on mice. The 4 epitopes (gp100.280, gp100.476, Melan-A/MART.1,27, Melan-A/MART-1.32, and Tyrosinase.1) which did not elicit CTL responses all fall into the weak and non CTL-inducer groups when administered as synthetic peptide in IFA with or without helper peptide. In mice assayed 17 weeks after injection of the polyepitope, 4 out of the five CTL responses could still be documented (data not shown). This suggests that memory CTL can be elicited using pCMV-B10 DNA polyepitope immunization.

### EXAMPLE 6

Comparative Immunogenicity of HIV 1-Derived, HLA-A2.1-Restricted Epitopic Peptides

The immunogenicity of 14 demonstrated (45, 46, 47, 48, 49, 10, 50, 51, 52) and 2 potential (47) HIV 1-derived, HLA-A2.1-restricted epitopic peptides of optimal length, listed in Table 5, was comparatively evaluated by infecting, s.c., groups of 6 HHD mice with peptides emulsified in IFA. Splenocytes were in vitro restimulated and the induced cytolytic activities evaluated in a classical ⁵¹Cr release assay using HHD-transfected RMA-S cells as targets loaded wit relevant or control (Inf, G9L) peptides.

The results are illustrated in Table 6. Considering the number of responder mice and the strength of their responses, the epitopic peptides were classified as strong (S9L, I9V), intermediate (T9V, L10V, A9M, V11V, V9L, L9V, K9L, R9V), or inefficient (E9V, P9L, E11Q, E10L, K9L/T, P10., A9L) immunogens. A similar evaluation was done co-injecting a I-A^{b} restricted CD4 epitopic peptide (HBV core T13L). Whereas some responses were enhanced, some other (S9L) were abolished, and, globally (as illustrated in Table 6), no significant improvement was observed.

TABLE 5
List of the HIV 1-derived CD8 epitopic peptides assayed

| | | | Frequency among | |
|---|---|---|---|---|
| Proteins of origin* | | CD8 epitopic peptides | HIV 1 isolates (%)** | References |
| GAG | p17 (77-85) | SLYNTVATL (S9L) (SEQ ID NO:25) | 39.6 | 15 |
| | p24 (19-27) | TLNAWVKVV (T9V) (SEQ ID NO:26) | 62.5 | 16 |
| | p24 (212-221) | EMMTACQGV (E9V)****(SEQ ID NO:27) | 95.8 | 17 |
| POL | (79-88) | LLDTGADDTV (L10V)****(SEQ ID NO:28) | 96.8 | 17 |
| | (188-196) | ALVEICTEM (A9M) (SEQ ID NO:29) | 46.2 | 18 |
| | (263-273) | VLDVGDAYFSV (V11V) (SEQ ID NO:30) | 84.9 | 17 |
| | (334-342) | VIYQYMDDL (V9L) (SEQ ID NO:31) | 84.9 | 19 |
| | (464-472) | ILKEPVHGV (I9V) (SEQ ID NO:32) | 68.8 | 20 |
| | (576-584) | PLVKLWYQL (P9L) (SEQ ID NO:33) | 87.1 | 18 |
| | (669-679) | ESELVNQIIEQ (E11Q) (SEQ ID NO:34) | 33.3 | 18 |
| | (671-680) | ELVNQIIEQL (E10L) (SEQ ID NO:35) | 33.3 | 18 |
| | (956-964) | LLWKGEGAV (L9V) (SEQ ID NO:36) | 98.9 | 16 |
| ENV | gp120 (120-128) | KLTPLCVSL (K9L) (SEQ ID NO:37) | 8.9*** | |
| | gp120 (120-128) | AKTPLCVTL (K9L/T) (SEQ ID NO:38) | 79.4 | 21 |
| | gp41 (260-268) | RLRDLLLIV (R9V) (SEQ ID NO:39) | 0.3*** | |
| NEF | (134-143) | PLTFGWCFKL (P10L) (SEQ ID NO:40) | 0.1 | 22 |
| | (188-196) | AFHHVAREL (A9L) (SEQ ID NO:41) | | 23 |

*Numeration is based on the amino-acid sequence of the HIV 1 WEAU clone 1.60 (Genbank Accession Number U21135.) Note however that the WEAU sequence may not be always identical to that of the reactive peptide and simply indicates its location in the viral proteins.
**Frequencies were calculated from the HIV Molecular Immunology Database (53) including the calculation all isolates irrespective of their clade appartenance.
***Barnaba, personal communication.
****Potential epitopes with highly affinity for HLA-A2.1 molecules.

TABLE 6
Cytolytic response of HHD mice injected with HIV
I-derived CD8 epitopic peptides emulsified in IFA

| | | without helper peptide^{a} | | With helper peptide^{b} | |
|---|---|---|---|---|---|
| | | Responder/ | Maximum specific | Responder/ | Maximal specific |
| CD8 epitopic peptides | | tested mice | lysis (%) | Tested mice | lysis (%) |
| GAG | S9L | 4/5 | 86, 60, 46, 26 | 0/6 | |
| | T9V | 2/6 | 22, 19 | 3/5 | 77, 50, 37 |
| | E9V | 0/6 | | 0/6 | |
| POL | L10V | 1/6 | 48 | 0/6 | |
| | A9M | 3/6 | 67, 15, 14 | 1/6 | 31 |
| | V11V | 3/6 | 57, 18, 16 | 2/6 | 30, 20 |
| | V9L | 1/6 | 34 | 1/6 | 57 |
| | I9V | 4/6 | 52, 35, 29, 16 | 5/6 | 94, 94, 87, 76, 35 |
| | P9L | 0/6 | | 0/6 | |
| | E11Q | 0/6 | | 0/6 | |
| | E10L | 0/6 | | 0/6 | |
| | L9V | 3/6 | 52, 22, 13 | 0/67 | |
| ENV | K9L | 3/6 | 18, 13, 10 | 4/6 | 56, 50, 34, 16 |
| | K9L(T) | 0/6 | | 1/6 | 27 |
| | R9V | 1/6 | 14 | 0/6 | |
| NEF | P10L | 0/6 | | 0/6 | |
| | A9L | 0/6 | | 0/6 | |

^{a}Mice were injected s.c. at the base of the tail with 100 μg of peptide emulsified in IFA. Seven days later, splenocytes were restimulated for 5 days in vitro with syngenic LPS-induced lymphoblasts pulsed with the immunizing peptide and tested in classical ⁵¹Cr-release assay against either relevant or control (Inf.m.G9L) peptide-pulsed HHD-transfected RMAS cells.
^{b}Same legend as in ^{a}, except that mice are co-injected with 50 μg of CD8 and 140 μg of CD4 HBV core T13L epitopic peptides.

### EXAMPLE 7

Relative Affinity for HLA-A2.1 Molecules and Stabilizing Capacities of the HIV 1-derived Epitopic Peptides

TAP-deficient T2 cells were incubated overnight with synthetic peptides and the amount and stability of cell surface HLA-A2.1 peptide/complexes evaluated in an indirect immunofluorescence assay as detailed in the Materials and Methods section.

Relative affinities (RA, the ratio of the concentrations of tested versus reference peptide, I9V, needed to reach equal amounts of cell surface expressed HLA-A2.1 molecules) and stabilizing capacities (the time, following removal of the peptides from the culture medium, for a 50% decay of cell surface stabilized HLA-A2.1 peptide complexes; DC50) are given for each peptide in Table 7. The lower the RA, the higher the relative affinity; the longer the DC50, the higher the stabilizing capacity.

As a rule, the peptides which were able to induce CTL responses exhibited significant relative affinity and stabilizing capacity, whereas the non-immunogenic peptides were both poor binders and poor stabilizers.

TABLE 7
Relative affinity and stabilizing capacity of HLA-A2.1 molecules of wild-
type and Tyrosine (P1)-substituted HIV 1-derived CD8 epitopic peptides

| | | Wild-type | Tyrosine substituted |
|---|---|---|---|
| CD8 epitopic peptides | | peptides RA*/DC** | peptides RA/DC |
| GAG | S9L | 5.5/>6 h | 5/>6 h |
| | T9V | 5.5/3.5 h | 5.5/>6 h |
| | E9V | 21/<2 h | 1.75/>6 h |
| POL | L10V | 10/<2 h | 1.5/4 h |
| | A9M | 5/2 h | 1.75/>6 h |
| | V11V | 4.5/2 h | 2.5/3.5 h |
| | V9L | >100/ND | 15/<2 h |
| | I9V | 1/5 h | ND |
| | P9L | >100/ND | 15/<2 h |
| | E11Q | >100/ND | >100/ND |
| | E10L | >100/ND | 10/<2 h |
| | L9V | 7.5/>6 h | 5/>6 h |
| ENV | K9L | 1.35/>6 h | 0.7/>6 h |
| | K9L/T | 0.65/>6 h | 0.4/>6 h |
| | R9V | >100/ND | >100/ND |
| NEF | P10L | >100/ND | 5/ND |
| | A9L | >100/ND | 7/>6 h |
| TAP⁻, HLA-A2.1⁺ T2 cells were incubated overnight with various concentrations of tested and reference (I9V) peptides. | | | |
| *Relative affinity (RA) is the ratio of the concentrations of tested versus reference peptides needed to reach 20% of the maximal amount of stabilized molecules as defined with high concentrations of reference peptide. | | | |
| **Half-life of stabilized peptide-HLA-A2.1 complexes (DC 50) was evaluated following T2 cells and peptide (100 μg/ml) after overnight incubation by measuring at time intervals (0, 2, 4, 6, 8 h) the amount of residual cell surface peptide-HLA-A2.1 complexes following peptide removal, T2 cells being maintained at 37° C. in the presence | | | |
| # of brefeldine A to block cell surface export of neo-synthesized HLA-A2.1 molecules. In all these experiments, the amount of stable peptide-HLA-A2.1 complexes was evaluated by indirect immunofluorescence and FACS analysis. ND, no done. | | | |

### EXAMPLE 8

Relative Affinities for HLA-A2.1 Molecules and Stabilizing Capacities of P1 Tyrosine Substituted Peptides

Substitution in P1 of wild-type residues by an aromatic residue (a favorable secondary anchor residue at the P1 position for HLA-A2.1 molecules (53)) has been reported in the I9V epitopic peptide case to enhance its immunogenicity (54). Similar improvements have been generalized to a series of 30 human cancer HLA-A2.1 restricted epitopic peptides (55). We therefore evaluated whether we could enhance both the affinity and stabilizing capacity of the HIV 1-derived epitopic peptide by replacing in P1 the wild-type residue by a tyrosine.

The results presented in Table 7 indicate that, with only few exceptions (E11Q, A9L(F)), this is indeed the case.

### EXAMPLE 9

Cytolytic Response of HHD Mice Injected with P1 Tyrosine Substituted Peptides

Mice were injected s.c. with the substituted peptides emulsified in IFA. Following in vitro restimulation with the substituted peptides, splenocytes were assayed in a classical ⁵¹Cr release assay against HHD-transfected, RMA-S cells loaded with either P1 tyrosine substituted, wild-type, or control (Inf, G9L) peptides.

The results are presented in Table 8. As a rule, significant improvements of the cytolytic responses were observed, both in terms of number of responder mice and strength of the responses. Notably, significant cytolytic responses were induced against Y/P9L and Y/P10L peptides, which were non-immunogenic in their wild-type configuration. Most interestingly, the

TABLE 8
Cytolytic response of HHD mice injected with tyrosine
(P1) substituted peptides and cross-recognition of
wild-type HIV I-derived epitopic peptides

| | | | | Cross- | |
|---|---|---|---|---|---|
| P1 | | | | recognition | |
| substituted | | | | of w.t. peptide | |
| (Y) epitopic | | Responder/ | | (% of maximal | |
| peptides | | tested mice* | Maximal lysis* | lysis)** | |
| GAG | Y/S9L | 4/6 | 55, 54, 34, 31 | 4/4 | 85% |
| | Y/T9V | 4/5 | 87, 62, 47, 26 | 4/4 | 85% |
| | Y/E9V | 1/6 | 47 | 1/1 | 53% |
| POL | Y/L10V | 0/6 | | | |
| | Y/A9M | 2/6 | 40, 30 | 1/2 | 33% |
| | Y/V11V | 0/6 | | | |
| | Y/V9L | 4/6 | 21, 17, 17, 16 | 4/4 | 105% |
| | Y/I9V | NT | | | |
| | Y/P9L | 3/6 | 96, 63, 55, 11 | 4/4 | 53% |
| | Y/E11Q | 0/6 | | | |
| | Y/E10L | 0/6 | | | |
| | Y/L9V | 1/6 | 30 | 1/1 | 43% |
| ENV | Y/K9L | 5/6 | 73, 60, 56, 33, 23 | 5/5 | 80% |
| | Y/K9L/T | 3/6 | 28, 27, 21 | 3/3 | 76% |
| | Y/R9V | 1/6 | 64 | 1/1 | 25% |
| NEF | Y/P10L | 4/6 | 90, 75, 37, 25 | 4/4 | 48% |
| | Y/A9L | 0/6 | | | |
| *Mice were injected s.c. at the base of the tail with 100 μg of tyrosine-substituted peptide emulsified in IFA. Seven days later, splenocytes were restimulated for 5 days in vitro with syngeneic LPS-induced lymphoblasts pulsed with the immunizing peptide and tested in a classical ⁵¹Cr-release assay against either tyrosine-substituted or control (inf.m.G9L) peptide-pulsed HHD-transfected RMAS cells. | | | | | |
| **Cross-recognition of wild-type peptide-pulsed HHD-transfected RMA-S cells was tested for each responder mouse. The number of mice cross-recognizing the wild-type peptide versus the number of mice recognizing the corresponding tyrosine-substituted immunizing peptide are given. The levels of cross-lysis, expressed in % correspond to the ratio of the sum of maximal lysis observed with the responder mice tested against | | | | | |
| # wild-type peptide pulsed target cells divided by the sum of maximal lysis obtained when tested against the corresponding, immunizing, tyrosine-substituted peptide pulsed targets. | | | | | |

induced responses were, in almost all cases, able to cross-lyse target cells loaded with the corresponding wild-type peptides. The level of cross-lysis ranged between 25 and 105%. Higher levels of cross-lysis were observed when poor binder/poor stabilizer peptides (e.g.,, P10L, data not shown) were further added in the culture medium during the cytolytic assay.

One exception was seen. The V11V peptide, in its tyrosine substituted form, was unable to induce cytolytic responses. Based on these results, either wild-type or tyrosine substituted peptides were selected for further immunizations. Wild-type peptides were selected if they were of equal or higher immunogenicity than their tyrosine substituted homologs.

### EXAMPLE 10

Cross-Recognition of the Main HIV 1 Natural Variants

Whereas some epitopic peptides tested are highly conserved among HIV 1 isolates (E9V, L10V, V11V, V9L, L9V, P9L), some others (S9L, T9V, A9M, P10L) are not. Therefore, we tested, in the four latter cases, whether the elicited CTL responses by the selected wild-type or tyrosine substituted immunizing peptides could cross-recognize natural variants of a significant representation among HIV 1 isolates.

Mice were immunized and spleen cells were restimulated in vitro with S9L, Y/P10, Y/T9V, or A9M synthetic peptides as indicated in the legend of Table 6. Cytolytic activity was evaluated against peptide-loaded HHD-transfected RMA-S cells using Inf.m.G9L peptide-loaded cells as a negative control. Sequence and frequency among HIV 1 isolates of the epitopic variants: S9L (39.6%); S9L/F (P3, Y→F, 18.8%); S9L/V (P8, T→V, 7.3%); P10L/F P→F, 19.3%); P10L/W (P1, P→W, 6.1%); T9V (62.5%); T9V/I (P9, V→I, 34%); A9M (46.2% A9M/T (P3, V→T, 9.7%); A9M/TK (P3, V→T; P7, T→K, 6.5%), A9M/TD (P3, V→T; P7, T→D, 6.5%).

The results, which are illustrated in FIG. 4, indicate that cross-recognition is generally observed. The cytolytic responses induced by S9L, Y/P10L, Y/T9V, and A9M cross-lysed significantly all main natural variants, whereas cross-recognition was not observed.

### EXAMPLE 11

Polyepitopic Immunization

A polyepitopic motif, without amino acid spacers, was introduced in the pre-S2 segment of the middle S Hepatitis B protein. The expression was controlled by an early CMV promoter. HHD mice were injected i.m. with pCMV-B10 recombinant DNA and splenocytes from individual mice were restimulated in vitro twice by each of the 13 epitopic peptides inserted in the polyepitope. Cytolytic activity was evaluated in a ⁵¹Cr release assay using wild-type and control peptide loaded HHD-transfected RMA-S target cells.

More specifically, the mice were injected i.m. with 100 μg of pCMV.B10 recombinant DNA encoding an HIV 1-derived polyepitope. Twelve days later, splenocytes from individual mice were restimulated in vitro twice for 5 days with synthetic peptides analogous to those included in the polyepitope, as detailed in the Materials and Methods section. Cytolytic assays were performed using HHD-transfected RMA-S cells loaded with corresponding synthetic peptides in their wild-type configuration. Inf.m.G9L loaded cells were used as a negative control.

The results are illustrated in FIG. 5. With the exception of L9V, all epitopic peptides included in the polyepitope induced specific cytolytic responses with significant recognition of the wild-type peptides. Globally, the same hierarchy in terms of immunogenicity was observed following immunizations with both polyepitopic DNA and purified epitopic peptides in IFA.

### REFERENCES

The entire disclosure of each of the following references is relied upon and incorporated by reference herein.

1. Melief, C. J., Tumor eradication by adoptive transfer of cytotoxic T lymphocytes. Adv. Cancer Res. 1992. 58: 143-175.

2. van der Bruggen, P., Traversari, C., Chomez, P., Lurquin, C., DePlaen, E., VandenEynde, B., Knuth, A. and Boon, T., A gene encoding an antigen recognized by cytolytic T lymphocytes on a human melanoma. Science 1991. 254: 1643-1647.

3. Rosenberg, S. A., Yang, J. C., Schwartzentruber, D. J., Hwu, P., Marincola, F. M., Topalian, S. L., Restifo, N. P., Dudley, M. E., Schwarz, S. L., Speiss, P. J., Wunderlich, J. R., Parkhurst, M. R., Kawakami, Y., Seipp, C. A., Einhorn, J. H. and White, D. E., Immunologic and therapeutic evaluation of a synthetic peptide vaccine for the treatment of patients with metastatic melanoma. Nat. Med. 1998. 4: 321-327.

4. Arnold, B. and Hämmerling, G. J., MHC class-I transgenic mice. Ann. Rev. Immunol. 1991.9: 297-322.

5. Kalinke, U., Arnold, B. and Hämmerling, G., Strong xenogeneic HLA response in transgenic mice after introducting an α3 domain into HLA-B27. Nature 1990. 348: 642-644.

6. Wentworth, P. A., Vitiello, A., Sidney, J., Keogh, E., Chesnut, R. W., Grey, H. and Sette, A., Differences and similarities in the A2.1-restricted cytotoxic T cell repertoire in humans and human leukocyte antigen-transgenic mice. Eur. J. Immunol. 1996. 26: 97-101.

7. Vitiello, A., Marchesini, D., Furze, J., Sherman, L. A. and Chesnut, R. W., Analysis of the HLA-restricted influenza-specific cytotoxic T lymphocyte response in transgenic mice carrying a chimeric human-mouse class I Major Histocompatibility Complex. J. Exp. Med 1991. 173:1007-1015.

8. Pascolo, S., Bervas, N., Ure, J. M., Smith, A. G., Lemonnier, F. A. and Pérarnau, B., HLA-A2.1-restricted education and cytolytic activity of CD8⁺ T lymphocytes from β2 microglobulin (β2 m) HLA-A2.1 monochain transgenic H-2D^{b} β2 m double knockout mice. J. Exp. Med. 1997. 185: 2043-2051.

9. Kawakami, Y. and Rosenberg, S. A., Human tumor antigens recognized by T-cells. Immunol. Res. 1997. 16: 313-339.

10. Tsomides, T. J., Walker, B. D. and Eisen, H. N., An optimal viral peptide recognized by CD8⁺ T cells binds very tightly to the restricting class I major histocompatibility complex protein on intact cells but not to the purified class I protein. Proc. Natl. Acad. Sci. USA 1991. 88: 11276-11280.

11. Milich, D. R., Hughes, J. L., McLachlan, A., Thornton, G. B. and Moriarty, A., Hepatitis B synthetic immunogen comprised of nucleocapsid T-cell sites and an envelope B-cell epitope. Proc. Natl. Acad. Sci. USA 1988. 85: 1610-1614.

12. Bednarek, M. A., Sauma, S. Y., Gammon, M. C., Porter, G., Tamhankar, S., Williamson, A. R. and Zweerink, H. J., The minimum peptide epitope from the influenza virus matrix protein. Extra and intracellular loading of HLA-A2. J. Immunol. 1991. 147: 4047-4053.

13. Sette, A., Vitiello, A., Reherman, B., Fowler, P., Nayersina, R., Kast, W. M., Melief, C. J., Oseroff, C., Yuan, L., Ruppert, J., The relationship between class I binding affinity and immunogenicity of potential cytotoxic T cell epitopes. J. Immunol. 1994. 153: 5586-5592.

14. van der Burg, S. H., Visseren, M. J., Brandt, R. M., Kast, W. M. and Melief, C. J., Immunogenicity of peptides bound to MHC class I molecules depends on the MHC-peptide complex stability. J. Immunol. 1996. 156: 3308-3314.

15. Vasilakos, J. P. and Michael, J. G., Herpes simplex virus class I-restricted peptide induces cytotoxic T lymphocytes in vivo independent of CD4+ T cells. J. Immunol. 1993. 150: 2346-2355.

16. Ossendorp, F., Mengede, E., Camps, M., Filius, R. and Melief, C. J., Specific T helper cell requirement for optimal induction of cytotoxic T lymphocytes against major histocompatibility complex class II negative tumors. J. Exp. Med. 1998. 187: 693-702.

17. Huang, A. Y., Golumbek, P., Ahmadzadeh, M., Jaffee, E., Pardoll, D. and Levitsky, H., Role of bone marrow-derived cells in presenting MHC class I-restricted tumor antigens. Science 1994. 264: 961-965.

18. Mayordomo, J. I., Zorina, T., Storkus, W. J., Zitvogel, L., Celluzzi, C., Falo, L. D., Melief, C. J., Ildstad, S. T., Kast, W. M., Deleo, A. B., Bone marrow-derived dendritic cells pulsed with synthetic tumour peptides elicit protective and therapeutic antitumour immunity. Nat. Med 1995.1: 1297-1302.

19. Layton, G. T., Harris, S. J., Myhan, J., West, D., Gotch, F., Hill-Perkins, M., Cole, J. S., Meyers, N., Woodrow, S., French, T. J., Adams, S. E. and Kingsman, A. J., Induction of single and dual cytotoxic T-lymphocyte responses to viral proteins in mice using recombinant hybrid Ty-virus-like particles. Immunology 1996. 87: 171-178.

20. Le Borgne, S., Mancini, M., Le Grand, R., Schleef, M., Dormont, D., Tiollais, P., Riviere, Y. and Michel, M. L., In vivo induction of specific cytotoxic T lymphocytes in mice and rhesus macaques immunized with DNA vector encoding an HIV epitope fused with hepatitis B surface antigen. Virology 1998. 240: 304-315.

21. Deres, K., Schild, H., Wiesmuller, K. H., Jung, G. and Rammensee, H. G., In vivo priming of virus-specific cytotoxic T lymphocytes with synthetic lipopeptide vaccine. Nature 1989. 342: 561-564.

22. Thomson, S. A., Sherritt, M. A., Medveczky, J., Elliott, S. L., Moss, D. J., Fernando, G. J., Brown, L. E. and Suhrbier, A., Delivery of multiple CD8 cytotoxic T cell epitopes by DNA vaccination. J. Immunol. 1998. 160: 1717-1723.

23. Gao, G. F., Tormo, J., Gerth, U. C., Wyer, J. R., McMichael, A. J., Stuart, D. I., Bell, J. I., Jones, E. Y. and Jakobsen, B. K., Crystal structure of the complex between human CD8alpha(alpha) and HLA-A2. Nature 1997. 387: 630-634.

24. Wilson, R. K., Lai, E., Concannon, P., Barth, R. K. and Hood, L. E, Structure, organization and polymorphism of murine and human T-cell receptor alpha and beta chain gene families. Immunol. Rev. 1988. 101: 149-172.

25. Momburg, F., Roelse, J., Howard, J. C., Butcher, G. W., Hämmerling, G. J. and Neefjes, J. J., Selectivity of MHC-encoded peptide transporters from human, mouse and rat. Nature 1994.367: 648-651.

26. Falk, K., Rötzschke, O., Stevanovic, S., Jung, G. and Rammensee, H. G., Allele-specific motifs revealed by sequencing of self peptides eluted from MHC molecules. Nature 1991. 351: 290-296.

27. Shirai, M., Arichi, T., Nishioka, M., Nomura, T., Ikeda, K., Kawanishi, K., Engelhard, V. H., Feinstone, S. M. and Berzofsky, J. A., CTL responses of HLA-A2.1-transgenic mice specific for hepatitis C viral peptides predict epitopes for CTL of humans carrying HLA-A2.1. J. Immunol. 1995. 154: 2733-2742.

28. Song, W., Kong, H. L., Carpenter, H., Torii, H., Granstein, R., Rafii, S., Moore, M. A. and Crystal, R. G., Dendritic cells genetically modified with an adenovirus vector encoding the cDNA for a model antigen induce protective and therapeutic antitumor immunity. J. Exp. Med. 1997.186:1247-1256.

29. Specht, J. M., Wang, G., Do, M. T., Lam, J. S., Royal, R. E., Reeves, M. E., Rosenberg, S. A. and Hwu, P., Dendritic cells retrovirally transduced with a model antigen gene are therapeutically effective against established pulmonary metastases. J. Exp. Med. 1997. 186: 1213-1221.

30. Porgador, A., Snyder, D. and Gilboa, E., Induction of antitumor immunity using bone marrow-generated dendritic cells. J. Immunol. 1996. 156: 2918-2926.

31. Burns, N. R., Gilmour, J. E., Kingsman, S. M., KIngsman, A. J. and Adams, S. E., Production and purification of hybrid Ty-VLPs. Mol. Biotechnol. 1994.1:137-145.

32. Inaba, K., Inaba, M., Deguchi, M., Hagi, K., Yasumizu, R., Ikehara, S., Muramatsu, S. and Steinman, R. M., Granulocytes, macrophages, and dendritic cells arise from a common major histocompatibility complex class II-negative progenitor in mouse bone marrow. Proc. Natl. Acad. Sci. USA 1993. 90: 3038-3042.

33. Pantaleo, G., C. Graziosi and A. S. Fauci. Virologic and immunologic events in primary HIV infection. Springer semin. Immunopathol. 1997. 18:257.

34. Koup, R. A., J. T. Safrit, Y. Cao, C. A. Andrews, G. M. Leod, W. Borkowsky, C. Farthing and D. D. Ho. Temporal association of cellular immune responses with the initial control of viremia in primary human immunodeficiency virus type 1 syndrome. J. Virol. 1994. 68:4650.

35. Borrow, P., H. Lewicki, B. H. Hahn, G. M. Shaw and M. B. A. Oldstone. Virus-specific CD8+cytotoxic T lymphocytes activity associated with control of viremia in primary human immunodeficiency virus type 1 infection. 1994. J. Virol. 68:6103.

36. Pantaleo, G., J. F. Demarest and T. Schacker. The qualitative nature of the primary immune response to HIV infection is a prognosticator of disease progression independent of the initial level of plasma viremia. Proc. Natl. Acad. Sci. USA. 1997. 94:254.

37. Buseyne, F., M. Burgard, J. P. Teglas, E. Bui, C. Rouzioux, M. J. Mayaux, S. Blanche and Y. Riviere. Early human immunodeficiency virus (HIV)-specific cytotoxic T lymphocytes (CTL) and disease progression in children born to HN-infected mothers. AIDS Res. Hum. Retroviruses. 1998. 14:1435.

38. Schmitz, J. E., M. J. Kuroda, S. Santra, V. G. Sasseville, M. A. Simon, M. A. Lifton, P. Racz, K. Tenner-Racz, M. Dalesandro, B. J. Scallon, J. Ghrayeb, M. A. Forman, D. C. Montefiori, E. P. Rieber, N. L. Letvin and K. A. Reimann. Control of viremia in simian immunodeficiency virus infection by CD8+lymphocytes. Science. 1999. 283:857.

39. Dean, M., M. Carrington, C. Winkler, G. A. Huttley, M. W. Smith, R. Allikmets, J. Goedert, S. P. Buchbinder, E. Vittinghoff, E. Gomperts, S. Donfield, D. Vlahov, R. Kaslow, A. Saah, C. Rinaldo, R. Detels and S. J. O'Brien. Genetic restriction of HIV-1 infection and progression to AIDS by a deletion allele of the CKR5 structural gene. Science. 1996. 273:1856.

40. Dalod, M., M. Dupuis, J.-C. Deschemin, C. Goujard, C. Deveau, L. Meyer, N. Ngo, C. Rouzioux, J.-C. Guillet, J.-F. Delfraissy, M. Sinet and A. Venet. Weak anti-HIV CD8+ T cell effector activity in HIV primary infection. J. Clin. Invest. 1999. In press.

41. Mellors, J. W., L. A. Kingsley and C. R. Rinaldo. Quantitation of HIV-1 RNA in plasma predicts outcome after seroconversion. Ann. Intern. Med. 1995. 122:573.

42. Musey, L., J. Hughes, T. Schacker, T. Shea, L. Corey. Cytotoxic T cell responses, viral load and disease progression in early HIV-1 infection. N. Eng. J. Med. 1997. 337:1267.

43. Tanchot, C., F. A. Lemonnier, B. Pérarnau, A. A. Freitas and B. Rocha. Differential requirements for survival and proliferation of CD8 naive or memory T cells. Science. 1997. 276:2057.

44. Man, S., J. P. Ridge and V. Engelhard. Diversity and dominance among TCR recognizing HLA-A2.1⁺ Influenza matrix peptide in human class I transgenic mice. J. Immunol. 1994. 153:4458.

45. Johnson, R. P., A. Trocha, L. Yang, G. P. Mazzara, D. L. Panicali, T. M. Buchanan and B. D. Walker. HIV-1 gag-specific cytotoxic T lymphocytes recognize multiple highly conserved epitopes. Fine specificity of the gag-specific response defined by using unstimulated peripheral blood mononuclear cells and cloned effector cells. J. Immunol. 1991. 147:1512.

46. Parker, K. C., M. A. Bednarek, L. K. Hull, U. Utz, B. C. H. J. Zweerink, W. E. Biddison and J. E. Coligan. Sequence motifs important for peptide binding to the human MHC class I molecules, HLA-A2. J. Immunol. 1992. 149:3580.

47. van der Burg, S. H., M. R. Klein, C. J. van de Velde, W. M. Kast, F. Miedema and C. J. Melief. Induction of a primary human cytotoxic T lymphocyte response against a novel conserved epitope in a functional sequence of HIV-1 reverse transcriptase. AIDS. 1995. 9:121.

48. Haas, G., A. Samri, E. Gomard, A. Hosmalin, J. Duntze, J. M. Bouley, H. G. Ihlenfeldt, C. Katlama and B. Autran. Cytotoxic T-cell responses to HIV-1 reverse transcriptase, integrase and protease. AIDS. 1998. 12:1427.

49. Harrer, E., T. Harrer, P. Barbosa, M. Feinberg, R. P. Johnson, S. Buchbinder and B. D. Walker. Recognition of the highly conserved region in the human immunodeficiency virus type 1 reverse transcriptase by HLA-A2-restricted cytotoxic T lymphocytes from an asymptomatic long-term non progresser. J.Inf. Dis. 1996. 173:476.

50. Dupuis, M., S. K. Kundu and T. C. Merigan. Characterization of HLA-A*0201-restricted cytotoxic T-cell epitopes in conserved regions of the HIV type 1 gp160 protein. J. Immunol. 1995. 155:2232.

51. Haas, G., U. Plikat, P. Debré, M. Lucchiari, C. Katlama, Y. Dudoit, O. Bonduelle, M. Bauer, H. Ihlenfeldt, G. Jung, B. Maier, A. Meyerhans and B. Autran. Dynamics of viral variants in HIV-1 Nef and specific cytotoxic T lymphocytes in vivo. J. Immunol. 1996. 157:4212.

52. Hadida, F., G. Haas, G. Zimmermann, A. Hosmalin, R. Spohn, A. Samri, G. Jung, P. Debré and B. Autran. CTL's from lymphoid organs recognize an optimal HLA-A2 restricted and HLA-B52 restricted nonapeptide and several epitopes in the C-terminal region of 1-HV-1 Nef. J. Immunol. 1995. 154:4174.

53. B. Korber, J. Moore, C. Brander, R. Koup, B. Haynes and B. Walker. Eds. HIV Molecular Immunology Database. 1998. Publisher, Los Alamos National Laboratory, Theoretical Biology and Biophysics, Los Alamos, N. Mex. Publication number LAUR 99-586.

54. Ruppert, J., J. Sidney, E. Celis, R. T. Kubo, H. M. Grey and A. Sette. Prominent role of secondary anchor residues in peptide binding to HLA-A2.1 molecules. Cell. 1993. 74:929.

55. Tourdot, S., A. Scardino, E. Saloustrou, D. A. Gross, S. Pascolo, P. Cordopatis, F. A. Lemonnier and K. Kosmatopoulos. A general strategy to restore immunogenicity of nonimmunogenic low HLA-A2.1 affinity peptides and discover cryptic tumor and virus epitopes of vaccines interest. 1999. Submitted for publication.

56. Cole, G. A., T. L. Hogg and D. L. Woodland. The MHC class I-restricted T cell responses to Sendai virus infection in C57BL/6 mice:a single immunodominant epitope elicits an extremely diverse repertoire of T cells. Int. Immunol. 1994. 6:1767.

57. Romero, P., N. Gervois, J. Schneider, P. Escobar, D. Valmori, C. Pannetier, A. Steinle, T. Wolfel, D. Lienard, V. Brichard, A. van Pel, F. Jotereau and J. C. Cerottini. Cytolytic T lymphocyte recognition of the immunodominant HLA A*0201 restricted Melan-A/MART-1 antigenic peptide in melanoma. J. Immunol. 1997. 159:2366.

58. Madden, D. R., D. N. Garboczi,and d. C. Wiley. The antigenic identity of peptide-MHC complexes: a comparison of the confirmations of five viral peptides presented by HLA-A2. Cell. 1993. 75:693.

59. Woodberry, T., J. Gardner, L. Mateo, D. Eisen, J. Medveczky, I. A. Ramshaw, S. A. Thomson, R. A. Ffrench, S. L. Elliott, H. Firat, F. A. Lemonnier and A. Suhrbier. Immunogenicity of a human immunodeficiency virus (HIV) polytope vaccine containing multiple HLA A2 HIV CD8⁺ cytotoxic T-cell epitopes. J. Virol. 1999. 73:5320.

What is claimed is:

1. A polynucleotide comprising:
a part of the coding sequence of the middle glycoprotein of the hepatitis B virus (HBV) into which is inserted a polynucleotide comprising a nucleic acid sequence coding for at least one viral, fungal, bacterial, or tumor epitope of an antigen, wherein the epitope induces a cellular response when administered to an individual.

2. The polynucleotide according to claim 1, wherein the polynucleotide comprises:
a part of the preS2 sequence of the HBV genome, into which is inserted a polynucleotide comprising a nucleic acid sequence coding for at least one tumor, viral, bacterial, or fungal epitope or a tumor, viral, bacterial, or fungal antigen; and
a nucleotide sequence coding for the surface antigen of HBV.

3. The polynucleotide according to claim 2, wherein said at least one epitope comprises 1 to 30 epitopes, inclusive, wherein none, some, or all of the epitopes are the same.

4. The polynucleotide according to claim 1, wherein said at least one epitope comprises 1 to 30 epitopes, inclusive, wherein none, some, or all of the epitopes are the same.

5. The polynucleotide according to claim 4, wherein one or more of the epitopes are mutated due to a mutation in the DNA encoding the epitopes.

6. A composition comprising the polynucleotide according to claim 1, wherein expression of at least part of the polynucleotide in vivo induces a cellular immune response against at least one tumor, viral, bacterial, or fungal specific antigen or tissue specific antigen, and all the self mutated or self-expressed proteins.

7. The composition according to claim 6, wherein the cellular immune response is induced in vivo against an HIV antigen.

8. The composition according to claim 7, wherein the HIV antigen is an envelope, gag, pol, or nef protein.

9. A vector comprising:
an early CMV promoter, preS2 and S nucleotide sequences encoding preS2 and S antigens of HBV;
nucleotide sequences derived from the genome HBV containing postranscriptional regulatory elements (PRE) and allowing nuclear export of RNA corresponding to nucleotide 1,151 to nucleotide 1,684 of the HBV genome;
signal sequences for polyadenylation of messenger RNAs of HBV located at position 1,921 to 1,955 of the HBV genome; and
nucleotide sequences of tumor, viral, bacterial, or fungal epitopes or antigens surrounded up and down by alanine spacers.

10. The vector according to claim 9, wherein expression of at least some of the sequences of the vector induces an in vivo cellular or/humoral immune response.

11. The vector according to claim 9, further comprising nucleotide sequences encoding a B cell epitope fused to at least one viral, bacterial, fungal, or tumor sequence, wherein the B cell epitope sequences allow the detection of hybrid proteins.

12. A process for treating an individual in vivo, said process comprising:
constructing the polynucleotide according to claim 1,
injecting the polynucleotide into an individual; and,
optionally, testing or evaluating the cytotoxic responses in the individual's lymphocytes.

13. A composition comprising a hybrid protein, wherein the hybrid protein comprises a preS2-S protein sequence and a sequence of at least one viral, bacterial, fungal, or tumor antigen or epitope.

14. The composition according to claim 13, wherein the composition induces, in vivo, a CTL response against one or more of the antigens or epitopes present on the hybrid protein.

15. The composition according to claim 13, wherein the hybrid protein further comprises a tag B cell epitope.

16. The composition according to claim 13, wherein the viral epitope is an HIV epitope.

17. The composition according to claim 16, wherein the HIV epitope is present on an envelope, gag, pol, or nef protein.

18. Recombinant particles comprising the composition according to claim 13 and the small envelope protein of HBV.

19. A process for treating cells of a host, said process comprising contacting the recombinant particles according to claim 18 with the host's cells to create treated cells; and
optionally, reinjection of treated cells in the host.
